(19) United States
(12) Patent Application Publication
(10) Pub. No.: US 2007/0072233 A1
(43) Pub. Date: Mar. 29, 2007

(54) METHODS AND SYSTEMS FOR FACILITATING THE DIAGNOSIS AND TREATMENT OF SCHIZOPHRENIA

(76) Inventors: Pat Ressler Levitt, Pittsburgh, PA (US); Venkata Chowdari Kodavali, Pittsburgh, PA (US); Vishwajit Laxmikant Nimgaonkar, Pittsburgh, PA (US); Karoly Mirnics, Pittsburgh, PA (US)

Correspondence Address: REED SMITH LLP, P.O. BOX 488, PITTSBURGH, PA, 15230-0488, US

(21) Appl. No.: 11/608,562

(22) Filed: Dec. 8, 2006

Related U.S. Application Data
(62) Division of application No. 09/939,209, filed on Aug. 24, 2001, now Pat. No. 7,166,462.
(60) Provisional application No. 60/288,021, filed on May 1, 2001.

(51) Int. Cl. C12Q 1/68; G01N 33/53

(52) U.S. Cl. 435/6; 435/7.1

(57) ABSTRACT

A method of diagnosing, assessing susceptibility, and/or treating schizophrenia involving the observation of regulator of G-protein signaling 4 (RGS4) levels in a subject. Embodiments of the present invention include increasing RGS4 expression levels in the cortex, either by chemical means or by genetic complementation (e.g. gene therapy).

### CROSS-REFERENCE TO RELATED APPLICATIONS

This application is a divisional application of pending U.S. Ser. No. 09/939,209, filed Aug. 24, 2001, which claims priority under 35 U.S.C. 119(e) to U.S. provisional application Ser. No. 60/288,021, filed Aug. 24, 1999.

This invention was made with United States Government support in the form of Grant Nos. MH45156, MH01489, MH56242, MH53459, and MH45156 from the National Institute of Mental Health. The United States Government may have certain rights in the invention.

### FIELD OF THE INVENTION

The present invention relates generally to the field of neurological and physiological dysfunctions associated with schizophrenia. The invention further relates to the identification, isolation, and cloning of genes which, when mutated or varied, are associated with schizophrenia. The present invention also relates to methods for diagnosing and detecting carriers of the genes and to diagnosis of schizophrenia. The present invention further relates to the construction of animal models of schizophrenia.

### BACKGROUND OF THE INVENTION

Schizophrenia is a serious brain disorder that affects approximately 1% of the human population. The cause of this complex and devastating disease remains elusive, although genetic, nutritional, environmental, and developmental factors have been considered. A combination of clinical, neuroimaging, and postmortem studies have implicated the dorsal prefrontal cortex (PFC) as a prominent site of dysfunction in schizophrenia.

Schizophrenia is typically characterized as a disorder of thinking and cognition, as contrasted to other disorders of mental faculties, such as mood, social behavior, and those affecting learning, memory, and intelligence. Schizophrenia is characterized by psychotic episodes during which an individual may lose the ability to test reality or may have hallucinations, delusions, incoherent thinking, and even disordered memory. There are varying forms of schizophrenia differing in severity, from a schizotypal disorder to a catatonic state. A review of schizophrenia can be found in Principles of Neural Science, 3^{rd} ed., 1991, Kandel, Schwartz, and Jessel (Eds.), Connecticut: Appleton & Lange, pp.853-868; of which Chapter 55 is incorporated herein by reference.

Diseases of organ systems, such as those of the heart, lung, and kidney, are usually confirmed by tissue pathology. A demonstrable pathology includes identifying and defining a structural abnormality in the organ, along with an associated alteration in organ function. This type of diagnosis is also utilized in certain neurological diseases. However, there are few psychiatric disorders in which clinical manifestations and symptoms can be correlated with a demonstrable pathology. The majority of mental illnesses are evaluated by observing changes in key behaviors such as thinking, mood, or social behavior. These alterations are difficult to ascertain and nearly impossible to quantify. However, progress is being made in diagnosing mental illness and in determining the neuropathology of mental illnesses.

The Diagnostic and Statistical Manual of Mental Disorders, Third Edition (DSM-III-R) and the updated DSM-IV, published by the American Psychiatric Association, represent the progress made in providing a basis for objective and rigorous descriptive criteria for categories of psychiatric disorders. While the DSM-III-R is very thorough and detailed, it is also quite lengthy. Thus, the process of reviewing the categories and applying them to data from a patient is also very time-consuming and arduous. In addition, there is no mechanism by which a patient can be diagnosed either as having or being susceptible to schizophrenia prior to the expression of symptoms. Thus, there is a longstanding need for an easy and definitive method for diagnosing schizophrenia. A diagnostic tool that can be applied prior to the expression of symptoms would also have great utility, providing a basis for the development of therapeutic interventions.

There is strong evidence for a genetic linkage of schizophrenia. Historically, there have been a number of studies on monozygotic twins of schizophrenics that indicated that 30-50% of the twins also had schizophrenia. The fact that this number is not 100% indicates that there are other factors involved in this disease process that may protect some of these individuals from the disease. It is apparent from a number of studies that the patterns of inheritance in most forms of schizophrenia are more complex than the classical dominant or recessive Mendelian inheritance. Recently, locus 1q21-22, a chromosome region containing several hundred genes, has been strongly linked to schizophrenia as shown by Brzustowicz et al., Science 288, 678-82, 2000, which is hereby incorporated by reference.

Until the 1950's there were no specific, effective treatments for schizophrenia. Antipsychotic drugs were identified in the 1950's, and these drugs were found to produce a dramatic improvement in the psychotic phase of the illness. Reserpine was the first of these drugs to be used and was followed by typical antipsychotic drugs including phenothiazines, the butyrophenones, and the thioxanthenes. A new group of therapeutic drugs, typified by clozapine, has been developed and were referred to as “atypical” antipsychotics. Haloperidol has been employed extensively in the treatment of schizophrenia and is one of the currently preferred options for treatment. When these drugs are taken over the course of at least several weeks, they mitigate or eliminate delusions, hallucinations, and some types of disordered thinking. Maintenance of a patient on these drugs reduces the rate of relapse. Since there is no way of determining if an individual is susceptible to schizophrenia, it is currently unknown if these antipsychotic compounds are useful in the prophylactic treatment of schizophrenia.

Signal transduction is the general process by which cells respond to extracellular signals (e.g. neurotransmitters) through a cascade of biochemical reactions. The first step in this process is the binding of a signaling molecule to a cell membrane receptor that typically leads to the inhibition or activation of an intracellular enzyme. This type of process regulates many cell functions including cell proliferation, differentiation, and gene transcription.

One important mechanism by which signal transduction occurs is through G-proteins. Receptors on the cell surface are coupled intracellularly to a G-protein that becomes activated, when the receptor is occupied by an agonist, by binding to the molecule GTP. Activated G-proteins may influence a large number of cellular processes including voltage-activated calcium channels, adenylate cyclase, and phospholipase C. The G-protein itself is a critical regulator of the pathway by virtue of the fact that GTPase activity in the G-protein eventually hydrolyzes the bound GTP to GDP, restoring the protein to its inactive state. Thus, the G-protein contains a built-in deactivation mechanism for the signaling process.

Recently, an additional regulatory mechanism has been discovered for G-protein signaling that involves a family of mammalian gene products termed regulators of G-protein signaling, or RGS (Druey et al., 1996, Nature 379: 742-746 which is hereby incorporated by reference). RGS molecules play a crucial modulatory role in the G-protein signaling pathway. RGS proteins bind to the GTP-bound Gα subunits with a variable Gα specificity as a substrate. RGS molecules shorten the GTP binding of the activated Gα subunits by acting as GTPase activating proteins (GAPs), accelerating GTP hydrolysis by up to one hundred fold. By the virtue of this GAP action and by making available the GDP-bound Gα to re-attach to βγ dimers, RGS proteins shorten the duration of the intracellular signaling. RGS proteins are expressed in nearly every cell; however, they show a tissue-specific expression across the body and cell type-specific expression in the brain. For example, RGS4 is strongly expressed in the central nervous system, moderately expressed in the heart, and slightly expressed in skeletal muscle (Nomoto et al., 1997, Biochem. Biophys. Res. Commun. 241(2):281-287 which is herein incorporated by reference).

Several members of the G-protein signaling pathways, most located downstream of RGS4 modulation, have been implicated in schizophrenia. Gil, Gq and Golf messenger RNA (mRNA) and protein levels all have been reported to be altered in various brain regions of the schizophrenic subjects. Furthermore, changes in expression of adenylate cyclase, phospholipase C, and protein kinases, as well as DARPP (dopamine- and cAMP-regulated phosphoprotein) phosphorylation changes are expected to be influenced by RGS regulation of Gα signaling. In addition, RGS modulation changes are expected to have significant effects on the signal transduction effected by neurotransmitters including dopamine, serotonin, GABA, glutamate, and norepinephrine.

An additional genetic marker of schizophrenia has been identified by Meloni et al. (U.S. Pat. No. 6,210,879). These investigators found that an allele of the microsatellite HUNTH01 in the tyrosine hydroxylase gene correlated with the expression of schizophrenia. However, the allele only appears to be present in sporadic schizophrenias.

There has been a long-standing need for a definitive and easy method for diagnosing schizophrenia as well as for an effective treatment with minimal side effects. Further, a need has been recognized in connection with being able to detect schizophrenia prior to the expression of noticeable symptoms.

A need has been recognized in connection with overcoming the various limitations to the current implementation of a method for diagnosing and assessing the susceptibility to schizophrenia are addressed through the use of the current invention.

### SUMMARY OF THE INVENTION

In accordance with at least one embodiment of the present invention, there is provided a system and method for diagnosing and determining the susceptibility to schizophrenia.

In summary, one aspect of the present invention provides an isolated and substantially purified DNA sequence corresponding to SEQ ID NOS: 3, 4, 5, 6, 7, 8, and contiguous portions thereof.

Another aspect of the present invention is a polynucleotide sequence that is complementary to a sequence selected from the group consisting of SEQ ID NO: 3, SEQ ID NO: 4, SEQ ID NO: 5, SEQ ID NO: 6, SEQ ID NO: 7, SEQ ID NO: 8, and contiguous protions thereof.

A further aspect of the present invention is an expression system comprising a DNA sequence that corresponds to SEQ ID NO:3.

A yet further aspect of the present invention is a method for diagnosing schizophrenia in a human comprising obtaining a DNA sample comprising a RGS4 gene from a patient and detecting a variation in the RGS4 gene indicating schizophrenia.

A still further aspect of the present invention is a method for determining the susecptiblity to schizophrenia comprising obtaining from a patient a DNA sample comprising a RGS4 gene and detecting a variation in said RGS4 gene indicating susceptibility to schizophrenia.

An additional aspect of the present invention is a method for daignosing schizophrenia comprising obtaining from a patient to be tested for schizophrenia a sample of tissue, measuring RGS4 mRNA levels in said sample, and determing if there is a reduced level of RGS4 mRNA in the sample.

A still additional aspect of the present invention is a method of determing susceptibility to schizophrenia comprising obtaining from a patient to be tested for susceptibility to schizophrenia a sample of tissue, measuring RGS4 mRNA levels in said sample, and determing if there is a reduced level of RGS4 mRNA in the sample.

A yet further aspect of the present invention is A method of determining susceptibility to schizophrenia comprising obtaining from a patient to be tested for susceptibility to schizophrenia a sample of tissue, measuring RGS4 protein levels in said sample, and determining if there is a reduced level of RGS4 protein in the sample.

Yet another aspect of the present invention is A method of treating schizophrenia, said method comprising measuring RGS4 protein or mRNA levels in a patient, and altering said RGS4 protein levels to provide the patient with an improved psychiatric function.

Another aspect of the present invention is a kit for diagnosising schizophrenia in a patient, said kit comprising antibodies to RGS4, and a detector for ascertaining whether said antibodies bind to RGS4 in a sample.

Another aspect of the present invention is a kit for diagnosising schizophrenia in a patient, said kit comprising a detect of RGS4 transcript levels in a patient, and a standard to ascertain altered levels of RGS4 transcript in the patient.

A still further aspect of the present invention is the DNA sequence of SEQ ID NO: 3 containing variations as described in the text below.

A yet further aspect of the present invention is a transgenic mouse whose genome comprises a disruption of the endogenous RGS4 gene, wherein said disruption comprises the insertion of a transgene, and wherein said disruption results in said transgenic mouse not exhibiting normal expression of RGS4 protein.

A still additional aspect of the present invention is a transgenic mouse wherein a transgene comprises a nucleotide sequence that encodes a selectable marker.

These and other embodiments and advantages of the present invention will be better understood with reference to the following figures and detailed description.

### BRIEF DESCRIPTION OF THE DRAWINGS

The present invention and its presently preferred embodiments will be better understood by reference to the detailed disclosure hereinbelow and to the accompanying drawings, wherein:

FIG. 1A displays the design of microarray immobilized probes and in situ probes for RGS4, wherein numbers on the RGS4 nucleic acid fragments denote nucleotide position in relationship to the RGS4 mRNA, as currently in the NCBI database;

FIG. 1B is a pseudocolored intensity view of a single RGS4 feature from the 516 control/547 schizophrenic PFC comparison after a dual-fluorescent hybridization; both images represent the same spot under cy3 and cy5 excitation, respectively; the balanced cy3 signal intensity (c-control subject) was 6.2-fold brighter than the cy5 signal intensity (s-schizophrenic subject);

FIG. 1C displays changes in RGS expression in the PFC of schizophrenic and control subjects reported by cDNA microarray analysis;

FIG. 2A shows in situ hybridization results for PFC RGS4 expression levels which are decreased in 9 of 10 schizophrenic subjects;

FIG. 2B shows the in situ hybridization data from 10 PFC pairwise comparisons which were quantified using film densitometry;

FIG. 3A shows that 632 G-protein signalling-related genes were detected out of 1644 possible detections (274 genes/microarray×six microarrays);

FIG. 3B shows that 239 1q21-22 locus-related genes were detected out of 420 possible detections (70 genes/mircoarray×six microarrays); RGS4 contribution to the transcript distribution is denoted by a hatched bar;

FIG. 4A shows high power photomicrographs of VC tissue sections from the same matched pair of schizophrenic and matched control subjects represented in FIG. 2A, viewed under darkfield illumination;

FIG. 4B shows a graph of 10 supragranular VC SCH pairwise comparisons, in which schizophrenic subjects showed a comparably significant RGS4 transcript reduction to the PFC comparisons;

FIG. 4C shows high power photomicrographs of MC tissue sections from the same matched pair of schizophrenic and matched control subjects represented in FIG. 2A, viewed under darkfield illumination;

FIG. 4D shows a graph in which schizophrenic subjects across the same 10 subject pairs across the MC had comparably decreased RGS4 expression levels (mean=−34.2%, F_{1,15}=10.18; p=0.006) to VC and PFC;

FIG. 5 shows a scatter plot of relative RGS4 expression changes across the experimental groups.

FIG. 6 displays the genomic organization that is derived from available sequences for clone NT_{—}022030, as well as the sequence analyses presented here; five exons were identified from the coding sequence for RGS4 (approximately 8.5 kb); the critical RGS domain is encoded by exons 3 to 5; the SNPs that were analyzed are listed in the top panel; * (a small star) indicates SNPs identified by re-sequencing the RGS4 gene and ★ (a large star) indicates SNPs used for association analysis.

### DESCRIPTION OF THE PREFERRED EMBODIMENTS

The present invention focuses on the genetic underpinnings of schizophrenia. In the first phase of the research, cDNA microarrays were used to investigate potential alterations in transcript expression in six pairs of schizophrenic subjects. RGS4 was determined to be the most significantly and consistently changed transcript. In situ hybridization was also used to verify the microarray findings and to examine the regional and disease-related specificity of this change. Out of the several hundred genes on locus 1q21-22, the present studies indicate that RGS4 is a strong candidate for a major susceptibility gene on this locus. Genetic association and linkage studies were conducted using two samples independently in Pittsburgh and by the NIMH Collaborative Genetics Initiative. Using the Transmission Disequilibrium Test (TDT), significant transmission distortion was observed in both samples, albeit with different haplotypes. In support of the TDT results, increased sharing of alleles, identical by descent was observed for polymorphisms in this region among affected siblings of the NIMH cases, though associations were not observed when the cases were compared to a limited number of population-based controls. These analyses are consistent with the possibility that inheritable polymorphisms in the flanking untranslated regions (UTR) of the RGS4 gene confer susceptibility to schizophrenia.

Expression Studies

Two groups of human subjects, consisting of six and five pairs of schizophrenic and control subjects, were used in the present studies. Subject pairs were completely matched for sex (18 males and 4 females). The mean (±SD) difference within pairs was 4.6±3.5 years for age and 4.4±2.7 hours for post mortem interval (PMI). The entire group of schizophrenic and control subjects did not differ in mean (±SD) age at time of death (46.5±10.7 and 45.1±11.5 years, respectively), PMI (19.4±7.1 and 17.7±5.0 hours, respectively), brain pH (6.85±0.29 and 6.81±0.15, respectively), or tissue storage time at −80° C. (45.4±12.3 and 37.7±13.1 months, respectively) when the studies initiated. Nine of the schizophrenic subjects were receiving antipsychotic medications at the time of death, five had a history of alcohol abuse or dependence, and one died by suicide. Also studied were 10 subjects with major depressive disorder (MDD), each of whom were matched to one normal control subject. The MDD subject pairs were also completed matched for sex (18 males and 2 females). The mean (S.D.) difference within pairs was 1.2±1.4 years for age and 2.5±2.1 hours for PMI. The depressive and control subjects did not differ in mean (±S.D.) age at time of death (52.7±13.1 and 52.1±13.1 years, respectively), PMI (14.9±5.3 and 15.7±5.5 hours, respectively), brain pH (6.81±0.17 and 6.72±0.30), or tissue storage time at −80° C. (39.0±17.4 and 39.9±13.2 months, respectively). Two of the depressed subjects had a history of alcohol dependence, and six died by suicide. Two of the control subjects had also been matched to subjects with schizophrenia (685c, 604c). Consensus DSM-IIIR diagnoses were made for all subjects using data from clinical records, toxicology studies, and structured interviews with surviving relatives.

RGS4 Transcript Analysis

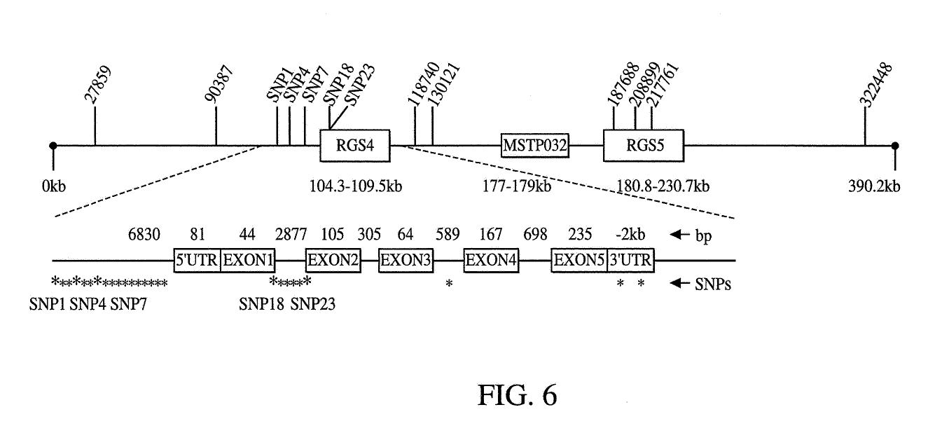

A Human Multiple Tissue Northern Blot (Clontech) and a ³²P-labeled cDNA probe were used to confirm the size of the RGS4 transcript reported previously (Druey, et al., 1996). However, our results reported the presence of single dark bands of ˜3 kB in lanes from multiple brain regions (whole cerebral cortex, frontal pole, occipital pole, temporal lobe), with much fainter or absent bands observed in lanes from other brain regions (cerebellum, medulla, spinal cord, putamen). Because the UniGene entry for the RGS4 cDNA (U27768) contained only the truncated transcript (800 bp), we designed custom PCR primers based on the BAC clone sequence containing the RGS4 gene (NT_{—}022030) to rapidly obtain the full-lenght RGS4 transcript sequence. For this analysis, mRNA from a control human brain was purified, DNased, and re-purified prior to first strand cDNA synthesis using Superscript II (Gibco) with an oligo dT primer. The resulting cDNA-mRNA mixture was diluted and used in a standard PCR reaction using AmpliTaq Gold (see above). All reaction products yielded single bright bands on 2% agarose/ethidium bromide-stained gels, and were subsequently purified and sequenced. Alignment of these sequences produced >99% identity matches with the BAC clone sequence containing RGS4. The 3′ UTR for RGS4 obtained in this manner also aligned >99% with a cDNA entry (AL137433.1) that contains both a poly A signal and a poly A attachment site, confirming that the human RGS4 transcript is 2949 bp without the poly A tail and includes a cDNA entry not previously associated with the human transcript in the NCBI database (see below; FIG. 6).

Microarray Experiments

Fresh-frozen human tissue was obtained from the University of Pittsburgh's Center for the Neuroscience of Mental Disorders Brain Bank. Area 9 from the right hemisphere was identified and isolated and sectioned into tubes at −24° C. as described previously by Glantz, L. A. and Lewis, D. A. in Arch Gen Psychiatry 54: 943-952, 2000, which is herein incorporated by reference. Total RNA and mRNA were isolated according to manufacturer's instructions using Promega (Madison, Wis.) kit #Z5110, RNAgents® Total RNA Isolation System and Qiagen (Valencia, Calif.) kit #70022, Oligotex mRNA Kits, respectively. The volume was adjusted using Microcon columns YM-30 #42409 to 50 ng/μl. The quality and purity of the mRNA used in the reverse transcription labeling reactions was evaluated by size distribution on a 10 non-denaturing agarose gel (>50% of mRNA smear over 1 kb; integrity of rRNA bands) and optical density (OD) measurements (260/280>1.80), respectively.

Sample Labeling, Microarrays, Hybridization, and Data Analysis

Labeling was performed at Incyte Genomics, Inc. (Fremont, Calif.). Two hundred nanograms of mRNA was reverse transcribed using cy3- or cy5-labeled fluorescent primers; appropriate matched control and schizophrenic sample pairs were combined, and hybridized onto the same UniGEM-V cDNA microarray. Each UniGEM-V array contained over 7,000 unique and sequence-verified cDNA elements mapped to 6,794 UniGene Homo sapiens annotated clusters found at the following NIH website: “http://www.ncbi.nlm.nih.gov/UniGene/index.html”. Hybridization and washing was performed using proprietary Incyte protocols. If a gene or expressed sequence tag (EST) was differentially expressed, the cDNA feature on the array bound more of the labeled probe from one sample than the other, producing either a greater cy3 or cy5 signal intensity. The microarrays were scanned under cy3-cys dual fluorescence, and the resulting images were analyzed for signal intensity. If the cy3 vs. cy5 signal intensity was within three fold, and the microarray detected spiked-in control standard less abundant than 1 copy in 50,000, the raw data were exported to a local SQL server database. On the server, the data were further analyzed using GemTools (Incyte's proprietary software) and MS-Excel 2000. Note that the operators performing the labeling, hybridization, scanning, and signal analysis were blind to the specific category to which each sample belonged.

Gene Expression Criteria

A gene was considered to be expressed if the DNA sample was successfully amplified by PCR, produced signal from at least 40% of the spot surface, and had a signal/background ratio over 5-fold for either the cy3 or cy5 probe. Based on Incyte's control hybridization studies (“http://www.incyte.com/reagents/gem/products.shtml/GEM-reproducibility.pdf”) and control experiments, array data reliability and reproducibility cutoffs were established as follows:
1. Genes were comparably expressed between the control and experimental samples if the cy3/cy5 ratio or cy5/cy3 ratio was <1.6.
2. Gene expression was changed between the two samples at the 95% confidence level (95% CL) if the cy3/cy5 or cy5/cy3 signal was 1.6-1.89.

3. Gene expression was changed between the two samples at the 99% confidence level (99% CL) if the cy3/cy5 or cy5/cy3 signal was >1.9. In the control experiments, <0.5% of the observations fell into this category.

Gene Group Analysis

Of the genes represented on the array, a G-protein group was created for data analysis, and included transcripts on the microarray for G-protein-coupled receptors (GPCR), heterotrimeric G-protein subunits, Ras proteins, regulator of G-protein signaling (RGS) molecules, and G-protein-dependent inward rectifying potassium channels (GIRKs), totaling 274 genes.

At least two genes, RGS4 (Unigene cluster Hs 227571) and RGS5 (Unigene cluster Hs 24950) were mapped to the cytogenetic band 1q21-22. In order to determine whether there is altered expression of multiple genes mapped to this locus, a 1q21-22 group was created from genes represented on the microarray locus. The 1999 NCBI database human 1q21-22 map is represented by 70 genes on the microarray, although some of them are not expressed in the central nervous system.

RGS4 Sequences

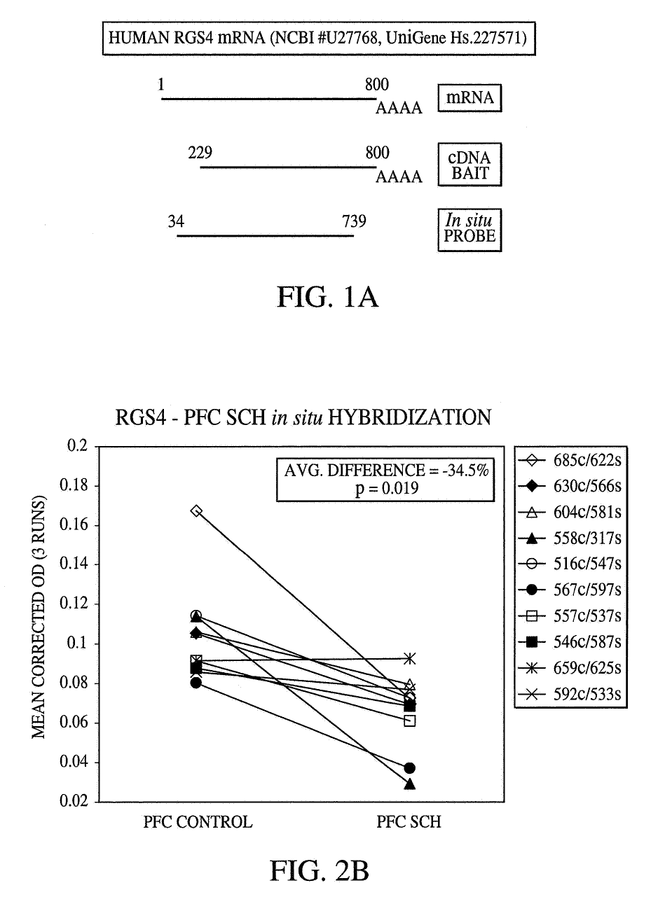

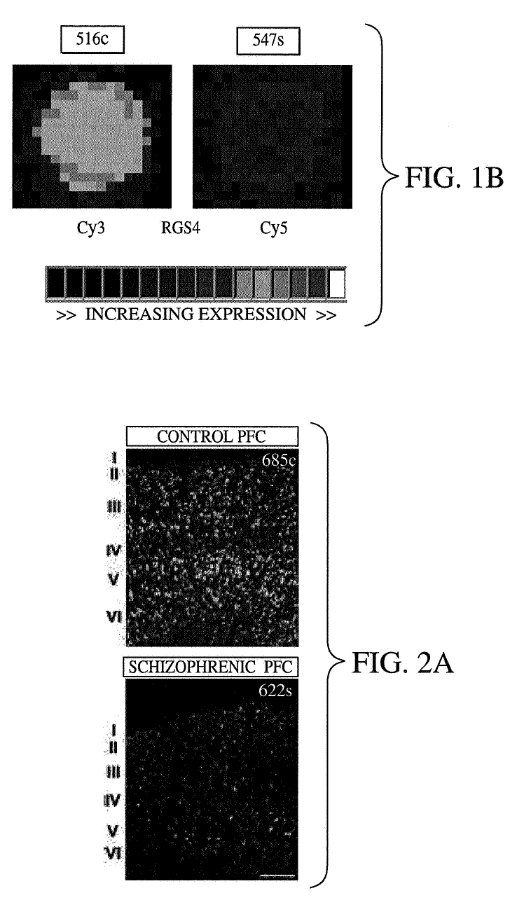

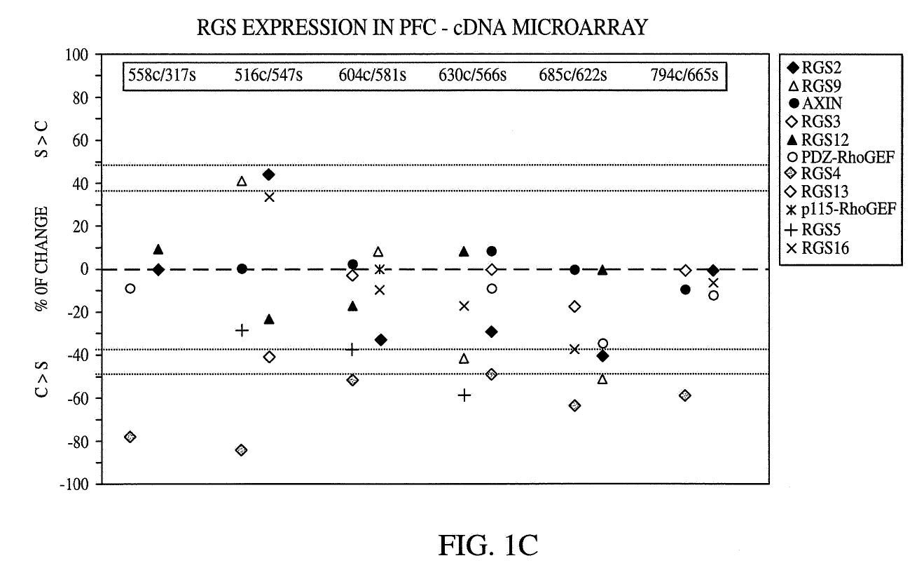

The RGS4 microarray immobilized probes sequence matched the entry in the NCBI database (accession number U27768, UniGene cluster Hs.227571). Of the 800 bp full-length mRNA, the double-stranded DNA microarray immobilized probe was complementary to the 3′ region of 571 nucleotides, as shown in FIG. 1A. The anti-sense, in situ hybridization probe was derived from the mRNA region spanning nucleotides 39-739, resulting in a 700 nucleotide long cRNA probe (see below). The RGS4 cDNA sequence, as determined from the complete mRNA coding sequence is listed as follows:

| | |
|---|---|
| gtacgctcaa agccgaagcc acagctcctc ctgccgcatt | 60 |
| tctttcctgc ttgcgaattc | |
| caagctgtta aataagatgt gcaaagggct tgcaggtctg | 120 |
| ccggcttctt gcttgaggag | |
| tgcaaaagat atgaaacatc ggctaggttt cctgctgcaa | 180 |
| aaatctgatt cctgtgaaca | |
| caattcttcc cacaacaaga aggacaaagt ggttatttgc | 240 |
| cagagagtga gccaagagga | |
| agtcaagaaa tgggctgaat cactggaaaa cctgattagt | 300 |
| catgaatgtg ggctggcagc | |
| tttcaaagct ttcttgaa9t ctgaatatag tgaggagaat | 360 |
| attgacttct ggatcagctg | |
| tgaagagtac aagaaaatca aatcaccatc taaactaagt | 420 |
| cccaaggcca aaaagatcta | |
| taatgaattc atctcagtcc aggcaaccaa agaggtgaac | 480 |
| ctggattctt gcaccaggga | |
| agagacaagc cggaacatgc tagagcctac aataacctgc | 540 |
| tttgatgagg cccagaagaa | |
| gattttcaac ctgatggaga aggattccta ccgccgcttc | 600 |
| ctcaagtctc gattctatct | |
| tgatttggtc aacccgtcca gctgtggggc agaaaagcag | 660 |
| aaaggagcca agagttcagc | |
| agactgtgct tccctggtcc ctcagtgtgc ctaattctca | 720 |
| cctgaaggca gagggatgaa | |
| atgccaagac tctatgctct ggaaaacctg aggccaaata | 780 |
| ttgatctgta ttaagctcca | |
| gtgctttatc cacattgtag cctaatattc atgctgcctg | 840 |
| ccatgtgtga gtcacttcta | |
| cgcataaact agatatagct tttggtgttt gagtgttcat | 900 |
| cagggtggga ccccattcca | |
| gtccaatttt cctaagtttc tttgagggtt ccatgggagc | 960 |
| aaatatctaa ataatggcct | |
| ggtaggtctg gattttcaaa gattgttggc agtttcctcc | 1020 |
| tcccaacagt tttacctcgg | |
| gatggttggt tagtgcatgt cacatgacat ccacatgcac | 1080 |
| atgtattctg ttggccagca | |
| cgttctccag actctagatg tttagatgag gttgagctat | 1140 |
| gatatgtgct tgtgtgtatg | |
| tctatgtgta tatattatat atacattaga cacacatata | 1200 |
| cattatttct gtatatagat | |
| gtctgtgtat acatatgtat gtgtgagtgt atgtatacac | 1260 |
| acacacacac acacacacac | |
| acacttttgc aagagtgatg ggaaagaccc taggtgctca | 1320 |
| taactagagt atgtgtatgt | |
| acttacatgg gtgttttgat ctctgttctt tcatactaca | 1380 |
| tttgaacagg gcaaaatgaa | |
| ctaactgcca tgtaggctaa gaaagaaatg ctaacctgtg | 1440 |
| gaaagttggt tttgtaaaat | |
| tccatggatc ttgctggaga agcatccaag gaacttcatg | 1500 |
| cttgatttga ccactgacag | |
| cctccacctt gagcactatt ctaaggagca aataccttag | 1560 |
| ctcccttgag ctggttttct | |
| ctgatggcac ttttgagctc ctaagctgcc agccttccct | 1620 |
| tcttttcctg ggtgctcagg | |
| gcatgcttat tagcagctgg gttggtatgg agttggcaga | 1680 |
| caggatgttc aacttaatga | |
| agaaatacag ctaaggcctt gccagcaaca cctgccgtaa | 1740 |
| gttactggct gagtgagggc | |
| atagaagtta aaggttactg tttttatcct ctatcctttt | 1800 |
| ttcctttcct gatcaaggtg | |
| ctcttctcat tttttcctga gaaccttagc catcagatga | 1860 |
| ggctccttag tttattgtgg | |
| ttggttgttt tttctttata atggctctgg gctatatgcc | 1920 |
| tatatttata aaccagcagc | |
| aggggaaaga ttatatttta taagagggaa caaattttca | 1980 |
| caatttgaaa agcccacata | |
| agttttctct tttaaggtag aatcttgtta atttcattcc | 2040 |
| aaacatcggg gctaacagag | |
| actggaggca tttcttttta ggctctgaga ctaaatgaga | 2100 |
| ggaaaagaaa agaaaaaaaa | |
| aatgattgtc taaccaattg tgagaattac tgtttgaaac | 2160 |
| ttttcaaggc acattgaaat | |
| acttgaaaac ttctcattta tgttatttat gatgttattt | 2220 |
| tgtacgtgtt attattatta | |
| tattgtttta taaatggagg tacaggatat cacctgaatt | 2280 |
| attaatgaat gcccaggaag | |
| taattttctt ctcattcttc taaaactact gcctttcaaa | 2340 |
| gtgcacacac acgcgtccac | |
| atacactgca ttcgttgctc cagtataaat tacatgcatg | 2400 |
| agcacctttc tggcttttaa | |
| gccaatataa tgggctgcaa aatgaagaca ccagagtgta | 2460 |
| tgcatacaaa tctcactgta | |
| ttaaagatgc aggttttcta attgtaccct tcttgtctct | 2520 |
| ctggcaatct tgcccttaat | |
| atccctggag ttcctcatca gtgtcatttt ctgttataca | 2580 |
| cagttccaca attttgtctc | |
| tagttgactt caaatgtgta actttattgg tcttgcccta | 2640 |
| ttataattgt catgactttc | |
| agattgtatc tgaactcaca gactgctgtc ttactaatag | 2700 |
| gtctggaagg tcacgctgaa | |
| tgagaagtaa attattttat gtaatacatt tttgagtgtg | 2760 |
| tttttcagtt gtatttccct | |
| gttatttcat cactatttcc aatggtgagc ttgcctgctc | 2820 |
| atgctccctg gacagaatac | |
| tccttccttt tgcatgcctg tttctatcat gtgcttgata | 2880 |
| ggcctcaaag ctaatgcttc | |
| cagtgaaaca cacgcatctt aataataagg gtaaataaac | 2934 |
| gctccatatg aaac | |

For purposes of the present invention, the RGS4 cDNA will be referred to as SEQ ID NO:1.

The 205 amino acid long sequence of RGS4, as determined and reported by Druey et al. in Nature, 379: 742-746 (1996) which is hereby incorporated by reference in its entirety, is listed as GenBank Accession number P49798 as follows:

MCKGLAGLPA SCLRSAKDMK HRLGFLLQKS DSCEHNSSHN
KKDKVVICQR
VSQEEVKKWA ESLENLISHE CGLAAFKAFL KSEYSEENID
FWISCEEYKK
IKSPSKLSPK AKKIYNEFIS VQATKEVNLD SCTREETSRN
MLEPTITCFD
EAQKKIFNLM EKDSYRRFLK SRFYLDLVNP SSCGAEKQKG
AKSSADCASL
VPQCA

The above amino acid sequence of RGS4 is referred to as SEQ ID NO: 2 for purposes of the present invention.

Untranslated regions upstream and downstream from the RGS4 coding region are identified in the context of the present invention as being relevant components of the RGS4 gene. The RGS4 coding sequence along with these sequences are found on NT_{—}022030 as described in greater detail below. This sequence is

| | |
|---|---|
| agttcaagac cagcctgagc aacatggtga aaccccatct | 60 |
| ctactaaaaa tacaaaatta | |
| gacaggcatg gtgatacacg cctgtaatcc cagctacttc | 120 |
| ggaggccgag gcaggagaat | |
| cacttgaacc tgctgggggt ggaggttgcg gggagcaaga | 180 |
| tcatgccatt gcactccagc | |
| ccaggcaaca agagcgaaat gtcatctcag aaaaaaaaaa | 240 |
| aggcatttta tatatatata | |
| tatatatata tacacacaca cacacatata tatatacaca | 300 |
| tatatataca catatataca | |
| tatatacaca tatatacaca tatatataca catacatatg | 360 |
| tacacatata tatacacata | |
| tgtatacaca tatatacaca tatatacaca catatataca | 420 |
| catatataca cacatatata | |
| cacatatata cacatatata cacatataca catatataca | 480 |
| catatataca tatatacaca | |
| tatatataat atacacacat atatatacac atatatacac | 540 |
| acatatatac acatatatac | |
| acatatatat acacatatat acacatatat acatatatac | 600 |
| acatatatat acatatatac | |
| acatatatac atatatacac atatatacat atatacacac | 660 |
| atatatacac atacatatac | |
| acacacatag atatacatat atatacacat atatatacgt | 720 |
| atatatatgt atatatatat | |
| gctccagagt tcataagagg tagcagttga ttaccactgg | 780 |
| ggatagagga aaagagagtt | |
| tgacagcagt gtattgtgag aaggacattt caggttgatg | 840 |
| gcaaatagta ggggaaatac | |
| ataaatgtgt aataaaacct atctgtaagg tagttaagaa | 900 |
| ggtaacacta tatatatata | |
| tagtgaaagc agtgtaaacc taaaggatgg gccaaggatt | 960 |
| taaatgttat agaagaatgg | |
| ctaagatgcc aaagctcagt gtatgtggca gaggcatggt | 1020 |
| gtagggtgtg tccaggttca | |
| tatattgcat taagtgtgag aacaccctgg agtatgaacc | 1080 |
| aagaaaatgc aaaagccaga | |
| agtgatggag gaaatgagac acaataatga agatattgag | 1140 |
| aggagggtgt gggcctagag | |
| tgaagctttt cgtgccagta cttcttttga aggcccagtt | 1200 |
| ctcttctctc tcgggggctc | |
| cttcatctct catagagtcc acagctttta agggccaaca | 1260 |
| cttgaggtca gcctggctct | |
| ctcatttgag ctggatagaa cattttagag caccatctat | 1320 |
| tcttcaagag gaagtttaaa | |
| aataaaagaa ccttgaagag gaaaaaatgt agacattcaa | 1380 |
| tctaaccttt tcattttact | |
| agccaaagct aaatagaatg caggttacct gtttttcagc | 1440 |
| caggcaccat catttcctaa | |
| ttgttataaa atttattatt attgttgtta ttattattat | 1500 |
| ttgccataag aagtttccca | |
| tatcctttta gtataacaaa aacacaattc acaagcatta | 1560 |
| taaaacccat ggtgtctaac | |
| tattaaaaaa attaagtgga acacacttgt cccagctact | 1620 |
| ggggaggctg aggagggagg | |
| atcacgtgat cccagggggt caaggttatg gagagctatg | 1680 |
| attgtgccac tgcactccag | |
| cctgggtgac agggaaagac cctgtctcta aaattttttt | 1740 |
| taaaaaaact aaactggttt | |
| tattacagag attctggaga cagctacaca taaaagggtg | 1800 |
| gtatgcctca tattagctac | |
| ccagggaggt ggaatgccaa cttaggtggt gtcaccacta | 1860 |
| ttaaaaatgc cccaaagcaa | |
| tcaaaactga gaacttcctg ggagcttagc attgtgcaaa | 1920 |
| agcagcacaa aacacttaaa | |
| caattcacag ttgtgttgga atgggaaggc ctggaaatat | 1980 |
| aaaccaaaga gtatattgtc | |
| taaattgata gagattacaa ttgcctgaaa gaaaaagttg | 2040 |
| acttttaact agaatgttca | |
| gagtaggttt acagaagaag ctcttaaact gggctccagt | 2100 |
| ggatttgtca atgctttgga | |
| agctggtggg gtgggagggt tggagggggc ataaaaagtc | 2160 |
| atgttggtat gctctgctca | |
| agtctccatt ctgtttcctt ttcctctttt caatgtcatg | 2220 |
| tcccattatt tcattatggg | |
| cttcccttta tccaggatca atatgccacc tcttggttgt | 2280 |
| cttttaccta cttctccacc | |
| tcactatgga atcgtccttg ggtagctcct gtgcttggga | 2340 |
| acctgcacgg gcacttttct | |
| gatgtcttga ttccagcttt actcctaaaa cttaaatgct | 2400 |
| gaggggccaa caccatggca | |
| gtggtaggga tgggaatggg ggtcttgtaa cacactacat | 2460 |
| aaactacacg aaataaacta | |
| catgaaactc aacatgtttg caagactcag ttcacatcca | 2520 |
| tgaggagctc atgcttctcc | |
| ctcctgctcc cctagcacac atgattatct ctatttggaa | 2580 |
| atgtttggca tttttggtga | |
| agtgaatggt tcaataactt tctccaccat cagaacaaaa | 2640 |
| gctctttaag gttagggatg | |
| ggatcataca cacttccctt gtccaagtcc ccatcacccc | 2700 |
| ttatctagac aattgctaca | |
| gtttcctaca cactcttcta acctcttgca gtctattttc | 2760 |
| ataaaacagc tagagaactt | |
| tgagatgtaa gtcaaaaaat agaacatgtc gctctttccc | 2820 |
| attgtttttg aaataaagtt | |
| caaccccctt accagggtca acaaggccct gcaatgattt | 2880 |
| ggtcctgtta aaaattcttt | |
| agccttaact catgctgttc ttccttacac tcactgcatt | 2940 |
| ctagccattg aggtttctat | |
| gcatcaaact ttttttggtc ccagcactgt gcacatcctt | 3000 |
| ctgggtagaa tgccccttga | |
| tttgtataat tagcacctcc ttcatcattt aggtcttagt | 3060 |
| ataactacta ccttcttaga | |
| gaagctctgc ttcttcatcc tataaaaaag taaaattcct | 3120 |
| taccctgtta ttttttaagt | |
| catccgtgtt tcattctgtt aaagttctta tcacaattta | 3180 |
| tcattatttt atttacagtc | |
| atgtgccaca taacaatgtt tcagtcaggg atagaacaca | 3240 |
| aatgtatctg gccccataat | |
| attataagct gagaaatttc tattaactag tgatatcgca | 3300 |
| gccatcataa gtgtaatgca | |
| ggacattacc ttttctatgt ttagatatgt tagatacaca | 3360 |
| aatatatttc attgtgttat | |
| aatttcctac agtattcagt acagtaacat gctgtacagg | 3420 |
| tttgtaacct aggagtaata | |
| ggctatacca tacagcttag gtgtgtagta ggctataacc | 3480 |
| atctaggttt gtgtaagtac | |
| attctatgat attcccacaa tgatgaaatc acctaactac | 3540 |
| acatttctca gaatgtttca | |
| ctgttgtgaa gtgacccatg actatatttt cctatatact | 3600 |
| tgatattttt gtgcatctgc | |
| ccatgagaat gtagtgtaag atcaaaggat gcaagaatgg | 3660 |
| gttctatcca gtatagtacc | |
| cactacactg gtggatgtca atatgtattt gttagattaa | 3720 |
| tatctcaaga atgagcacct | |
| ttctcagaca cataaaagat gctcaatata aaagtttgtt | 3780 |
| gaactgaacg ttattggcaa | |
| atgtaacatg atcggattta aagaggagcg aaacagaggt | 3840 |
| ctggctcaaa caccatactt | |
| ctagagtgca taagaggtag cagttgatta ccactggcga | 3900 |
| caggagaaaa aagagcttga | |
| ccgcagggta ctgtgaagac atttcaggtt gatggcacag | 3960 |
| aacaggggaa atacataaat | |
| gtgtgggaat attcagtggt ctgggatgac tacatagtag | 4020 |
| aatataatga agaaaagagt | |
| ggaagggaaa gatgaaaagt tggaatgggg atgaattatg | 4080 |
| aaagtaccag aatgttatgc | |
| taaggaatct agattttaaa atgtgagggc aaattgaagt | 4140 |
| [symbol] c [symbol] gggcacg ttacaaaact | |
| agaggtcata aagtttaccc taatttacca agatttccta | 4200 |
| gaggatctat aattggaatc | |
| cagatctgcc tctctgtaaa gttcaagcac tttccatgac | 4260 |
| accatactgt ttctttccac | |
| ctgcacaatg caaatgaact cttatgaaac tgctgtttct | 4320 |
| atcctgggct aaatgttgca | |
| gaaaaaagat ttaatctttg ggataaggct attttgggtt | 4380 |
| ttctcct [symbol] ct tcttgggaaa | |
| caaggttttc ttcccctggc taattaagtg tggtattgtt | 4440 |
| cttccaggga aatcagtgat | |
| gcatcacctg ctgctatcaa atgtcagggt tggagttcct | 4500 |
| gatttattgc atgtgcccac | |
| aaagcttggt gcaaagaatt ggacacattt cccaaaagta | 4560 |
| agacatactg ggaagtccct | |
| gtttaccttc ctggtataca gcatcctcca gccccatatc | 4620 |
| tttgcttttt agtcctaaaa | |
| [symbol] tcaataact gaactctcat tgatgtctag gccattgtag | 4680 |
| taaacaataa agaaggaggg | |
| aggcttctga caactgagag gaaattgtca tctgaagtgg | 4740 |
| tgcaagcaca gcctggggct | |
| gagccttggc ctacatcctg cccaagtgga ggatcagtg [symbol] | 4800 |
| cccatttaac atctggtaga | |
| actaaagaac gcaac [symbol] cctg ccacaatgac ttatttccct | 4860 |
| gcatttgata ccgtcaatcc | |
| ttgagaaatg ttttcttttg ttctccctga gcaaaggttg | 4920 |
| gaaaaatttg aaatttacct | |
| agagaccaca catagttcac atcctgctgt gtggctgaat | 4980 |
| gtctgcccc [symbol] cagtaggaaa | |
| cagttcttct aaagcctatt gtcaacaata ccttccagat | 5040 |
| gttagcattt tacaatttaa | |
| ggaacttaaa atag [symbol] cttca aactttttgc cagtttctct | 5100 |
| gatatccaat ctattctttt | |
| actctgcctc ccaagctttc tttctagaat gctaacctga | 5160 |
| tcggcttaag tacttgaact | |
| acctcttctc ctccattaac tacagagtaa attctggtct | 5220 |
| tcagagtaac aagaaacacc | |
| ctttagttct cagcatattc gtgcaccttc atttatctct | 5280 |
| ccttctctct caaagctgca | |
| gtaggggtga aaac [symbol] tgtga tacattttct cttccatcat | 5340 |
| aagggtcgca accaaaactc | |
| ctatagtaaa agacaggtta ataagagcaa aacctaacaa | 5400 |
| atttatttaa tcaaagtttt | |
| acatgacatg ggagtcttca gaaatgaaga cccaaagacc | 5460 |
| caggggaaac tgtctgtttt | |
| ttttgctgag gttcgatgaa gaatggatag catgtagcca | 5520 |
| tgtagattag acaaaaggat | |
| atgatctagt ggtaaaggac tcagggggaa acacagcaag | 5580 |
| gcctgtctat tcagattctt | |
| cttgatctct ctctctctat gtatagcatt ctttcctcct | 5640 |
| gagtatgggg caggactctt | |
| cttcaatgag ggtcttcaag ggagaaggga gaaagtggcc | 5700 |
| tttttagatt ttat [symbol] [symbol] cttg | |
| cttcggggaa gaggagttct agtttctatg acccatcttg | 5760 |
| gggaagagga attctggttt | |
| ctgtgacttg ctttcatgaa gaaagaggag taagaggcag | 5820 |
| gagggcagga gatggtcaga | |
| aagagacttg gctgcttctg agggcttccg ctctccttta | 5880 |
| gttccaagta cttcttagca | |
| taccaaagca ctatactttg gcatatggtt ttctgagctc | 5940 |
| taacactgca atcatgctaa | |
| actcctctat gaccttcaaa cattccactt gcttttattc | 6000 |
| tttatggttg tgatggcata | |
| gaggtcaata gcaaagaccc tggagtccca ctgtctgagc | 6060 |
| tggcataaca ttactaccac | |
| ttaatcaatg tgtaagctca ggtaagtact taagtcctct | 6120 |
| atgcttcatc tgtaaaatga | |
| gaatcattga agaacattct ctcaggatgg atcatgagga | 6180 |
| ataagtgaat taactggcat | |
| atagtgctta aaccagtgcc ttgctcagtt agtgacagat | 6240 |
| aaaatcatct gttattactg | |
| tgcccactat tgtgatgctc ttctcttctt tgtacaacga | 6300 |
| ctacatctct atttatcatt | |
| ttagggtctc cttgtgaaaa accactccag attcaaaaga | 6360 |
| ttgagtttaa tctctatcct | |
| ctgtgctttc ctggagtttt gtaaagtaaa tcttcacttg | 6420 |
| acatcatgga taggttcttg | |
| gaaactacaa cttcaagtga aaggacataa ctaaaccaat | 6480 |
| ttttttctca tcaacgttat | |
| aatgaaatgg cattgatgaa atgatggcat tcaaggacct | 6540 |
| gctgtacctt gtttcactta | |
| aagtcactgt ttccaataat ctattgatga cattgaggac | 6600 |
| ttactatata ataataaata | |
| tatatataat cgacgaaaca ggaatcaaac tgctaactct | 6660 |
| gctaactggt ctccctgctt | |
| ccacactctg cccactcatc tcagtctttc tttcacaaga | 6720 |
| gtcagaatga tcagatgaga | |
| cccctcctct gcttctgttt cttccatgga tttccactgc | 6780 |
| actctgataa agtccagcct | |
| cttgaccaca gcctacaaat ccttgcacga tctatcgttt | 6840 |
| acttttccat ctccttttat | |
| gctactttca tcttgttctc aattctctag ctatgctggc | 6900 |
| cccttcttgt tctttcccat | |
| ttttttttaa tttttaaaat ttgtatatat ttatgggtta | 6960 |
| taagtgaaat ctttttagat | |
| gcataggttg tatagtgata aaatcagggc ttttagggta | 7020 |
| ttcatcacct gaatgatgta | |
| cattgtaccc cttaagtaat ttctcaccat ccgctgactt | 7080 |
| cttgccccct gggtattcat | |
| cacctgaatg atgtgcattg taccccttaa gtaatttctc | 7140 |
| accatccgct gacttcttgc | |
| cccctgggta ttcatcacct gaatgatgtg cattgtaccc | 7200 |
| cttaagtaat ttctcaccat | |
| ccgctgactt cttgccccct catccttctg aggctccatt | 7260 |
| gtccatcatt ccacactcta | |
| catctatgtg tacacattat ttagctccta cttataagtg | 7320 |
| ataacatgca atatttgtct | |
| ttctgtgtct gtcttgtttt acttatgata atggccccca | 7380 |
| gttctatcta ggct [symbol] ctgca | |
| aaaggcatga tttcattctt ttttatggct atgttctttc | 7440 |
| ccaatttaga taaagaacac | |
| tcgcacttgc tcttacttct atttggaata ctaattccta | 7500 |
| ggcttcttgc attgctttct | |
| ccttctcacc catcaaatct cattttagat accacctctt | 7560 |
| caaagagggc tttcctgacc | |
| accttggctg aattagccct tcaccatctg attactctct | 7620 |
| agcacatcac ctgcccattt | |
| tattcatggt acaggtcaaa atctggaatc acctgatttg | 7680 |
| tttattttct gactccttct | |
| actgagatga aaactctact agagcggaga ttttatctgc | 7740 |
| ttgtatcagg tactgcttca | |
| aacagcacct gatacaga [symbol] t aggtggtcaa aagatatttc | 7800 |
| ttaaacaaat gaacaaataa | |
| aaagtagatc ttttgagagt aaagctcttc cacactacca | 7860 |
| gagtcattca ggaatgacaa | |
| atcatagaat aacagaattt gatgctttgt gcatatcaga | 7920 |
| gaaagaaggt ggaaggttgt | |
| caaggtatca tgatgtacca gtcctcgcct cctcaaacac | 7980 |
| aatctgcaag tcccacagtg | |
| aaaaagtaag ttaactcatg tgaagcgttt tacaaacact | 8040 |
| tttttaaaag tcttaaaact | |
| cctaagaaag caagatttaa tagtcaaaga agtgagtaaa | 8100 |
| catgaaatgc ctgaacagag | |
| taatgagcta agcacaaagt tagagacatg ttagttaata | 8160 |
| tgtcttgaaa gcagcagctc | |
| ctgctttcaa ggagcaagaa caaattgggc aagtgaacac | 8220 |
| tccttgaata aaatgtgtaa | |
| aattaatttt gggttatgtt ctatactgtg tataatagaa | 8280 |
| tgataaaaat tatttgacta | |
| gcactttgta gtttagaaat atctctattt acacagttta | 8340 |
| ccttatttga taagactgtt | |
| gagtgatggg atagcatggt ggacaatcca cataactgag | 8400 |
| tatcgagaca cctgtatctg | |
| gacccagctc tgttagtaag aagctgtaac ctcagcaagt | 8460 |
| cactttctct ttctgggtct | |
| ctatttcctt tttggtgaaa tgagagtgtt aggctagatt | 8520 |
| gcctttgaag tcccattttg | |
| tctttaaagt cccatctatt gcagtgattt atatttaact | 8580 |
| catgacaaat caggcttctc | |
| ttattctaag tgcaa [symbol] acat aaaactttta ttgtggaatt | 8640 |
| tcaggcatca gtaaatcttt | |
| ttgggtactc acttatgttc ctgaaatcaa tctatttgag | 8700 |
| tgatcactct tttaggtgcc | |
| caggtaaaca aagaaggcca tggtctttct ttgagtgacc | 8760 |
| ttctttccct tttaattagt | |
| ctgacctctt taatgtcagt tctgactgat tcatttccct | 8820 |
| ggtccatctt ccttggtctg | |
| agggccttcc tagtttcata ttgcacttca gttccttcca | 8880 |
| caccaccatc aaggatggct | |
| gtcaacattc atttgttcta tgttataatt caaggaaaag | 8940 |
| ttgcccagta gctaatccaa | |
| taaatgccct cttatgggcg gctagagact ttttcctata | 9000 |
| atttaaatgc atcttctgta | |
| gattatggtc cctccaccac tttacatttg tctgctgtct | 9060 |
| ccttgctctg ctagtcatgg | |
| aacgtgttgg tagtgggggc agtgtgggat gttcaagggc | 9120 |
| acgtattggg tagggccaca | |
| tatgggcatt gctttgtgcc attctttcta tatttttggt | 9180 |
| attttgcatc tcactggaac | |
| ccaactattt ttcatctctt ccacctaaac tatttgatgc | 9240 |
| ctctgtttct tatatataaa | |
| gtatagctca ctgtagccta tgatcaggaa cctatctgct | 9300 |
| ttctaaatga aagctgtttt | |
| ggtcagatct agcaattaat tcccttcttc cacttatagc | 9360 |
| tttcctctgt aactctggtg | |
| taggtatttg gtttatggct ataagatgtg aaacacctga | 9420 |
| atgattctgt ccatgcaggc | |
| atttcagttc atgatattgt atgtaaaaga tactgattgt | 9480 |
| ctaggtgttc agaaacacct | |
| atagggctta atattcttac aatcagtttg aaggctggtg | 9540 |
| atacgcaaag caaactacat | |
| atttttctgc ctgctctctc tctttctctc tacatctctc | 9600 |
| tttctttatc ttttgaaata | |
| tcagtttgga gacttagaat tacataagac ataaacccat | 9660 |
| ttgatataag aattgctgtg | |
| tatatttgct catctactcc ctcctttggt cctcgagctg | 9720 |
| ccggtttaga ctttttacag | |
| gacgcaggca tgtgaaggag aaactgtcag tgctaggctg | 9780 |
| aattctgttg ttaccaagat | |
| ttctagaaaa gtattcctca gtcaggttga ttacagatat | 9840 |
| agcaaatcta tttttcctag | |
| ggtagtttct gtatgctgcc gggcttataa ctgtctgtca | 9900 |
| tccagctatt t [symbol] tctccacc | |
| ttcttgtttg cataacaacc aaggcaactt ccgcaaatca | 9960 |
| ctgcgtggag acgatgatcc | |
| tg [symbol] cagctcc cttttggaaa tcgtgaggat cagatcttgg | 10020 |
| accatgtata atatgatgct | |
| tctaatccaa aagaggaaag gcattgggag tcagctccta | 10080 |
| agtaagctcc agaattcctg | |
| ctggtacttt tccttccagg aagcaacttc cttgatattt | 10140 |
| tttttttaca g [symbol] catatgaa | |
| taaaaactat attttgcagc attgtacact ttttttcctt | 10200 |
| ttctagaaat tctaaacctc | |
| tgacattggt ggagacattg agtacatttt ttcccatatc | 10260 |
| cctacttttc agaaggattt | |
| tctctgctcg ttcacttaac attgctgatg cgtcagtctt | 10320 |
| ttcttcctca tctctttcag | |
| gggctggaga ggcagaggga gacagaggag ctggtactgc | 10380 |
| agagcggtcg tctgattggc | |
| tggacggtcg tagctgggct ataaaagaga cccctacagg | 10440 |
| cttagcagga agacgctcag | |
| aggattctga caatatcttt accggagaag aggcaaagta | 10500 |
| cgctcaaagc cgaagccaca | |
| gctcctcctg ccgcatttct ttcctgcttg cgaattccaa | 10560 |
| gctgttaaat aagatgtgca | |
| aagggcttgc aggtctgccg gcttcttgct tgaggaggta | 10620 |
| agattgcttt cagccattaa | |
| ccatattaaa cttttggcta gactttctca gttatttaca | 10680 |
| tgttgtactt actaacctag | |
| ttctgtgcaa ttagaaacag tgtggtcagg agagcacgac | 10740 |
| tttctaactt tcctccaaga | |
| ctagctagat attgtgactt aagacatgtg ctccccaaat | 10800 |
| ttcagccctt atgtgttgtt | |
| ttgtgtgacc tcagttttga gaactgttct attctttaag | 10860 |
| ccaggtctaa gaaagctagt | |
| tttaattaag aagcgagatg aggtttgagg ctatgtacag | 10920 |
| tgatctgtaa tatctccatc | |
| tgtgattact actgctattt gagcatccct ggagtacata | 10980 |
| gaagcctggc tctgggcttt | |
| ctgattgtat gctacaactt gtttcaggaa aggtacccca | 11040 |
| gaatgaggtt tggctccatc | |
| atcagaaagg cacta [symbol] gctt tccgtgtggt ggtgcagtaa | 11100 |
| ctttcactct [symbol] tatgttctt | |
| ataag [symbol] aaat gttacaatga gatatgagtt ttaaagccag | 11160 |
| atcttcctta tctctctgcc | |
| ccatctctag ttcttgaagt gtctcatatg agtttggttg | 11220 |
| agaaatattg atcattacaa | |
| atcagttaat agttttgtag aagatctcat cttaaagaca | 11280 |
| ttgttttgtt aatatactcc | |
| cttgattttt ttaaaagacc ttacagacat acagctattc | 11340 |
| atttgttttt ggtttgttca | |
| aaaaaggtat aaagaaatgc attcagagaa agatcatata | 11400 |
| ttagccagtt gaaaattaaa | |
| cacaaaatga gtgcatatta cattacttaa tcttgcagtc | 11460 |
| aaaggtaaaa agtcaaccta | |
| aaggtatact acctgctttc ttatcgcact gcaaatagaa | 11520 |
| attaccacaa attttatttt | |
| ggaaataatc tcagaaaaca taatttttta tgtactatta | 11580 |
| aaacatttac tttccaaata | |
| ttctgtcatt caggagtatg gaagtatcga tggcttcttt | 11640 |
| aaaatgaagc aggagggtct | |
| ggcagagagt atctatgaaa taagttcctc tgaccttcac | 11700 |
| gcttaatttt ctgaatggag | |
| tggagcaaat tacttcaagc ttcacttaac ttgcatatga | 11760 |
| aatgaaccgt acaaaaatac | |
| aagagtgtca gga [symbol] aaagtt atgctctggt aaatattttg | 11820 |
| caaaacagat aaaagataat | |
| actagagcta tgtcctcaaa gagttaagca gctaatctaa | 11880 |
| ggaggtaaac tctatgtcag | |
| caggatgaac tgctcttccc tttcctcctc aataaattgc | 11940 |
| aaatcatcta gtccaacatc | |
| tttaccacca gtgcctgagg ctccagagga gccattgcct | 12000 |
| tctcaaggtc acataggtgg | |
| tgggtgagtt aggaccaaat ctagaattcc tgactccagt | 12060 |
| aacttctgaa gtcattttgt | |
| tttttatttt tatggtttta ttataagaat acttgctaag | 12120 |
| cacacttacc ccctgcattg | |
| attaataact ctaggatctc ag [symbol] t [symbol] gatcc agcacataga | 12180 |
| aatatgaatt cgtttctatt | |
| tggacttcat gatatattta cattatcacc ttggaatcac | 12240 |
| cctaacattc aggattgtat | |
| cttgttataa tcaaaaagga tgttgcatcc cctgaacagt | 12300 |
| catcagtcag ggaagcagag | |
| gagggaaagt aatcttgcga ggaagagaaa atactattta | 12360 |
| agggacagtc agagaacata | |
| atggaattca aactttctgg gaaaacctac atacataaat | 12420 |
| gtattagtgg ccatcctaaa | |
| tgtctttata tctttgaggc tttattttcc ctactccaaa | 12480 |
| tagacacatt tagttattca | |
| tttcttttaa aatggtattt ctctttttaa actatttctt | 12540 |
| gactttttta ataaaaagag | |
| atgcaagcaa gaggatattt aataaaaagt aagagagttg | 12600 |
| agcttaaggc ttattaaaag | |
| accccctttt tctagttagt caggagctct aatgtgccct | 12660 |
| ggctacctat taaatggtgg | |
| caataaactg gaagctcagt gatgactcta gcctgcttct | 12720 |
| cctaatagct gttaagcctc | |
| aaatgccctt tagagtgtgt atgtccttta aagtagctat | 12780 |
| taagaaggaa agcagcagca | |
| gcagatattg tctagaaaga agccccaaga agctgaggtt | 12840 |
| tcagcttggg catttgtttt | |
| cgccatccca tgctccattt ccctctgctg gaactgtgca | 12900 |
| cctcagtgta ttctccctct | |
| atacctcaca gcaggaactg cttgcccccc cccccccccc | 12960 |
| ccaacataca tggctggaac | |
| tgaatagact tttactttcc cgaggtgctt ctacagttcc | 13020 |
| ctctgccagc aggggaacag | |
| atggaaatag caatcacctg ccagaaggtg gcgtgcagca | 13080 |
| aggatgtgca tcttttgccg | |
| ctactgcttt ctgattccta aaaattactc agagatcact | 13140 |
| catgtgttca gtgattcagg | |
| ttctgttgaa gataccaaag atattcggtt ggtcaaaatg | 13200 |
| acgggcatat aaaggcttct | |
| caggtttctg aggtaaactg aagggtcaga attccagttg | 13260 |
| tggatgaagg aaatggtgtt | |
| atgactgcct caaggttttg tagcaagtca tagggaacca | 13320 |
| agaggaatct tgttttcctc | |
| agaggtcatg ccaactccaa ctcccgttcc ctaaactgtc | 13380 |
| tctgagccat agactagtaa | |
| tggactcttc aagctctacc attaggtatc ttttaaagaa | 13440 |
| agctggttat tactatttat | |
| tcattttttt ctcttctgtg cagtgcaaaa gatatgaaac | 13500 |
| atcggctagg tttcctgctg | |
| caaaaatctg attcctgtga acacaattct tcccacaaca | 13560 |
| agaaggacaa agtggttatt | |
| tgccagaggt aagagaaaag gccttggtga agatgtactt | 13620 |
| agtattaact atctgatgat | |
| ggggatgttc tgtgagaagg aacttgtgct cctagttaag | 13680 |
| ccagatttgg atcaagatag | |
| cctccatttt catggagatc ataactacat ttgaaatttc | 13740 |
| tatacattta gtgaaaaact | |
| gccctcatca ataacatatt ttgtcataac gatggaaaat | 13800 |
| aaaatctttg ccttcattca | |
| ggatcttaga tttcttgccc caattttttt accatggcat | 13860 |
| tccaattatt ctgtttctct | |
| ctattttttc tagagtgagc caagaggaag tcaagaaatg | 13920 |
| ggctgaatca ctggaaaacc | |
| tgattagtca tgaatgtaag tctgacagca acctgggatg | 13980 |
| aggtactctg gataagacaa | |
| gttatattat gctggtctaa tagaaactgc agcaaggcct | 14040 |
| ggcttctttc tgatgttcag | |
| actcaggaga ctctttaggt cttaaattca gtctgtttaa | 14100 |
| aattttaata tgccctagag | |
| ctttgtgata tacaatgaaa agtttatgca ggaaccatgt | 14160 |
| ggaaaaccat ctctctcatc | |
| acaaggaaaa acggaagaga gaaaaaaaat gataaatatc | 14220 |
| aataccttct tgcaaaatca | |
| atctcagttt ctctttccca aattgacctt ggtaattgat | 14280 |
| agctgcatag gcatttcaga | |
| agcaaaatac ttccttgaaa gaggcttcca acttgagtaa | 14340 |
| gaatcattag gtagaactgg | |
| gaaccactgg atatcaaaca cagatt [symbol] ggg ttacctgact | 14400 |
| ccaggtgact tgaaaaaagc | |
| aggggaaaaa gggattgctt gaatccatgc tttatccccc | 14460 |
| aagtacctca gctttatgtg | |
| aaatagcata tccaagaggc caaccagtgt gatgacaact | 14520 |
| gtggtccttt ctcctgtatc | |
| ataggtgggc tggcagcttt caaagctttc ttgaagtctg | 14580 |
| aatatagtga ggagaatatt | |
| gacttctgga tcagctgtga agagtacaag aaaatcaaat | 14640 |
| caccatctaa actaagtccc | |
| aaggccaaaa agatctataa tgaattcatc tcagtccagg | 14700 |
| caaccaaaga ggtaggtttt | |
| ttatggatac ataaaaattg tacgtattta tggagtatgt | 14760 |
| gtgatatttt gatacatgca | |
| tacaatgtga taacaatcaa atcagggcaa ttgctatata | 14820 |
| catatctcaa acatttatta | |
| tttctacgtg ttgagaacat tccaaatctc ctcttctagc | 14880 |
| tatcttaaaa tatacaataa | |
| actattgata actatatcac cctaatgtgc tatcaaacac | 14940 |
| tagaacctat tccctctacc | |
| caactttcta tctattcctt ctacccatta gccaacctga | 15000 |
| ccaaaaaggt aagcttttat | |
| ggcagagaac tctctggatc ttagtgaagg ttcctagaat | 15060 |
| agtggagctg actatcataa | |
| tcttgacaac cccaaataaa tcagtttttt aaaaaatctc | 15120 |
| ttttatccat gtggcttacc | |
| ataacctccc tgcatgaatt tttctgatga atctccccaa | 15180 |
| tttgttagac agaacagaag | |
| atcttgccct gctctctcta aagcagaaag gttcattctg | 15240 |
| aacctttcat actctctcac | |
| atgtgccaag gaggacccca atgtcacttt tgttttttgc | 15300 |
| ttctgaaata cagagggtgc | |
| actgccactt acaagtcact acaaagcata caggcttgca | 15360 |
| tcctcaacag ggatataggt | |
| ctaatgaagc cttggccttt gcccctcagg tgaacctgga | 15420 |
| ttcttgcacc agggaagaga | |
| caagccggaa catgctagag cctacaataa cctgctttga | 15480 |
| tgaggcccag aagaagattt | |
| tcaacctgat ggagaaggat tcctaccgcc gcttcctcaa | 15540 |
| gtctcgattc tatcttgatt | |
| tggtcaaccc gtccagctgt ggggcagaaa agcagaaagg | 15600 |
| agccaagagt tcagcagact | |
| gtgcttccct ggtccctcag tgtgcctaat tctcacctga | 15660 |
| aggcagaggg atgaaatgcc | |
| aagactctat gctctggaaa acctgaggcc aaatattgat | 15720 |
| ctgtattaag ctccagtgct | |
| ttatccacat tgtagcctaa tattcatgct gcctgccatg | 15780 |
| tgtgagtcac ttctacgcat | |
| aaactagata tagcttttgg tgtttgagtg ttcatcaggg | 15840 |
| tgggacccca ttccagtcca | |
| attttcctaa gtttctttga gggttccatg ggagcaaata | 15900 |
| tctaaataat ggcctggtag | |
| gtctggattt tcaaagattg ttggcagttt cctcctccca | 15960 |
| acagttttac ctcgggatgg | |
| ttggttagtg catgtcacat gacatccaca tgcacatgta | 16020 |
| ttctgttggc cagcacgttc | |
| tccagactct agatgtttag atgaggttga gctatgatat | 16080 |
| gtgcttgtgt gtatgtctat | |
| gtgtatatat tatatataca ttagacacac atatacatta | 16140 |
| tttctgtata tagatgtctg | |
| tgtatacata tgtatgtgtg agtgtatgta tacacacaca | 16200 |
| cacacacaca cacacacact | |
| tttgcaagag tgatgggaaa gaccctaggt gctcataact | 16260 |
| agagtatgtg tatgtactta | |
| catgggtgtt ttgatctctg ttctttcata ctacatttga | 16320 |
| acagggcaaa atgaactaac | |
| tgccatgtag gctaagaaag aaatgctaac ctgtggaaag | 16380 |
| ttggttttgt aaaattccat | |
| ggatcttgct ggagaagcat ccaaggaact tcatgcttga | 16440 |
| tttgaccact gacagcctcc | |
| accttgagca ctattctaag gagcaaatac cttagctccc | 16500 |
| ttgagctggt tttctctgat | |
| ggcacttttg agctcctaag ctgccagcct tcccttcttt | 16560 |
| tcctgggtgc tcagggcatg | |
| cttattagca gctgggttgg tatggagttg gcagacagga | 16620 |
| tgttcaactt aatgaagaaa | |
| tacagctaag gccttgccag caacacctgc cgtaagttac | 16680 |
| tggctgagtg agggcataga | |
| agttaaaggt tactgttttt atcctctatc cttttttcct | 16740 |
| ttcctgatca aggtgctctt | |
| ctcatttttt cctgagaacc ttagccatca gatgaggctc | 16800 |
| cttagtttat tgtggttggt | |
| tgttttttct ttataatggc tctgggctat atgcctatat | 16860 |
| ttataaacca gcagcagggg | |
| aaagattata ttttataaga gggaacaaat tttcacaatt | 16920 |
| tgaaaagccc acataagttt | |
| tctcttttaa ggtagaatct tgttaatttc attccaaaca | 16980 |
| tcggggctaa cagagactgg | |
| aggcatttct ttttaggctc tgagactaaa tgagaggaaa | 17040 |
| agaaaag [symbol] aa aaaaaaatga | |
| ttgtctaacc aattgtgaga attactgttt gaaacttttc | 17100 |
| aaggcacatt gaaatacttg | |
| aaaacttctc atttatgtta tttatgatgt tattttgtac | 17160 |
| gtgttattat tattatattg | |
| ttttataaat ggaggtacag gatatcacct gaattattaa | 17220 |
| tgaatgccca ggaagtaatt | |
| ttcttctcat tcttctaaaa ctactgcctt tcaaagtgca | 17280 |
| cacacacgcg tccacataca | |
| ctgcattcgt tgctccagta taaattacat gcatgagcac | 17340 |
| ctttctggct tttaagccaa | |
| tataatgggc tgcaaaatga agacaccaga gtgtatgcat | 17400 |
| acaaatctca ctgtattaaa | |
| gatgcaggtt ttctaattgt acccttcttg tctctctggc | 17460 |
| aatcttgccc ttaatatccc | |
| tggagttcct catcagtgtc attttctgtt atacacagtt | 17520 |
| ccacaatttt gtctctagtt | |
| gacttcaaat gtgtaacttt attggtcttg ccctattata | 17580 |
| attgtcatga ctttcagatt | |
| gtatctgaac tcacagactg ctgtcttact aataggtctg | 17640 |
| gaaggtcac [symbol] ctgaatgaga | |
| agtaaattat tttatgtaat acatttttga gtgtgttttt | 17700 |
| cagttgtatt tccctgttat | |
| ttcatcacta tttccaatgg tgagcttgcc tgctcatgct | 17760 |
| ccctggacag aatactcctt | |
| ccttttgcat gcctgtttct atcatgtgct tgataggcct | 17820 |
| caaagctaat gcttccagtg | |
| aaacacacgc atcttaataa taagggtaaa taaacgctcc | 17880 |
| atatgaaact atttgcttgg | |
| aaacacatta atgatccaga gacatgctat gagaaacatc | 17940 |
| agggtgtagg gtgactttag | |
| aaaaatactc atactgagtc tttaatccct cctgtgccag | 18000 |
| tgaactctgg gaaagaaagt | |
| acaaactgaa tattgtttat tctttagttc atgccactgc | 18060 |
| tctgcttggc tctactcata | |
| gaaccaaggc aatcttagct tcagagactg caaaacagat | 18120 |
| taagtgattt gcttgcagat | |
| tctcaatcaa ttttcaaggg atagagttca ccttccagag | 18180 |
| ccattctttt atttccagtt | |
| acccgcctgt ttgagagatg atagagcagt gggaaattga | 18240 |
| gagagttgaa aggagctata | |
| gattcttacc caaacttcaa aaatccttcc ctcccttttg | 18300 |
| ttaattctct ttcctggaaa | |
| agaggtcata aaatgttcac atcctcagta ataggccctg | 18360 |
| tgctgtgtct attatgtcat | |
| gagactccca tttcctgacc cttctttccc attgtaagag | 18420 |
| tagtagttac aaggtgttaa | |
| ggatagatga tcttcaacac ttttgagaaa tagatccatt | 18480 |
| tacggatctg gtaaaaacta | |
| tggaccgaac catcttttaa gaaaaaaatt cagagaggaa | 18540 |
| tctaaatttt gtgtgctttg | |
| aggggaaact ctcagaatct cccctcaaaa ctatcattct | 18600 |
| tctcttatac tatagatgtg | |
| tcagactctc actgggactg tatagttgct gctccctgta | 18660 |
| tttgataata tctatcaaga | |
| actgcagggt aattcaaagt cacgctatta gcagcaagtg | 18720 |
| tgagcagtgt tggtttcccc | |
| agtctctaca tccctcatcc tttctttctt ctttatggtt | 18780 |
| gtctattaaa gaaataaaaa | |
| aaaatattgg ctgaccgttt ttctgaagat aatgtatatc | 18840 |
| aaggaccacc ttttgaaaaa | |
| cactcattat tcgagaacaa agacacaaca tacgagaatc | 18900 |
| tctgggatac attcaaagca | |
| gtgtgtagag ggaaatttat agcactaaat gcccacaaga | 18960 |
| gaaagcagga aagatctaaa | |
| attgataccc taacatcaca attaaaagaa ctagaaaagc | 19020 |
| aagagcaaac acattcaaaa | |
| gctagcagaa gacaagaaat aactaagatc agagcagaac | 19080 |
| tgaaggaaat agagacacaa | |
| aaaacccttc aaaaaattaa tgaatccagg agctggtttt | 19140 |
| ttgaaaagat taacaaaatt | |
| gatagactgc tagcaagact aataaagaag aaaagagaga | 19200 |
| agaatcaaat agacacaata | |
| aaaaatgata aaggggatat caccaccgat cccacagaaa | 19260 |
| tacaaactac catcagagaa | |
| tactataaac acctctacgc aaataaacta gaaaatctag | 19320 |
| aagaaatgga taaattcctc | |
| gatacataca ccctcccaag accaaaccag gaagaagttg | 19380 |
| aatctctgaa tagaccaata | |
| acaggctctg aaattgaggc aataatcaat agcttaccaa | 19440 |
| ccaaaaaaag tccaggacca | |
| gatggattca cagctgaatt ctaccagacg tacaaagagg | 19500 |
| agctggtacc attccttctg | |
| aaactattcc aatcaataga aaaagaggga atcctcccta | 19560 |
| actcatttta tgaggccagc | |
| atcatcctga taccaaagcc tggcagagac acaaccaaaa | 19620 |
| aagagaattt tagaccaata | |
| tccttgatga acattgatgc aaaaatcctc aataaaatac | 19680 |
| tggcaaaccg aatccagcag | |
| cacatcaaaa agcttatcca ccatgatcaa gtgggtttca | 19740 |
| tccctgggat gcaaggctgg | |
| ttcaacatac gcaaatcaat aaatgtaatc cagcatataa | 19800 |
| acagaaacaa agacaaaaac | |
| cacatgatta tctcaataga tgcagaaaag gcatttgaca | 19860 |
| aaatttaaca actcttcatg | |
| ctaaaaactc tcaatcaatt aggtattgat gggacgtatc | 19920 |
| tcaaaataat aagcactatc | |
| tatgacaaac tcacagccaa tatcatactg aatgggcaaa | 19980 |
| aactggaagc attccctttg | |
| aaaacgggca caagacaggg atgccctctc tcaccactcc | 20040 |
| tattcaacat agtgttggaa | |
| gctctggcca gggcaattag gcaggagaag gaaataaagg | 20100 |
| gtattcaatt aggagaagag | |
| gaagtcaaat tgtccctgtt tgcagatgac atgattgtat | 20160 |
| atctagaaaa ccccatcgtc | |
| tcagcccaaa atctccttaa gctgataagc aacttcagca | 20220 |
| aagtctcagg atacaaaatc | |
| aatgtacaaa aatcacaagc actcttatac atcaataaca | 20280 |
| gacaaacaga gagccaaatc | |
| atgagtgaac tcccattcac | 20300 |

For purposes of the present invention, this DNA sequence will be referred to as SEQ ID NO:3. The location of the SNPs discussed further below is indicated by bold and larger font letters. Several additional sequences of DNA that are upstream from SEQ ID NO:3 are identified as relevant to the present invention. These DNA sequences are also found on NT_{—}022030 and are

| | |
|---|---|
| ggattaatca tgacaaaagt aatctaaatc tcgttaagac | 60 |
| tacttaatga tcaatctttc | |
| cctctgtttt ccctgactat agggaagtga attgccccaa | 120 |
| tccttctcta tcacccccct | |
| gcagccatgc caatgcctta cctctgttat attcagccat | 180 |
| aggggaagct tattctcata | |
| gaatcagggg ttggcatg [symbol] a gtcactagct attcttggtg | 240 |
| agactagtga agatgagtga | |
| aggaaaatat tgcataggtg aaatctcata ggcacaaata | 300 |
| ggtgtttgtg agagtaacaa | |
| taaaagaaag tcattcccat actctagtag atgactcatt | 360 |
| ttctcctcat tttttttttt | |
| tcaaggcgtt ctctacaacg gttaacctag taccaaaaat | 420 |
| ccttctcttt tttcttggac | |
| aaatcctgtt caagttagca tggcatttac tacgtccaag | 480 |
| acattgtcca gatgctgtgg | |

For purposes of the present invention, this DNA sequence will be referred to as SEQ ID NO:4.

| | |
|---|---|
| agagaaagaa aggcaggcag caaggagaaa aaacattttt | 60 |
| taaaaaaaga aaattaaaat | |
| ccatgtaatg tctgatatct gttctgctgt atgtgtagat | 120 |
| ctttccatat accaactcat | |
| tagccttatt ttacaggtga ggaaaatgag ac [symbol] gagagtc | 180 |
| cttcttactt gaccaagttc | |
| acacagcaag atcacacatg gtagaaccaa tgttagaacc | 240 |
| taggtgtata cttgctcatt | |
| caatatgtac aataattgca aaagtttcca taggtcttat | 300 |
| tatatatcag gcactataaa | |
| tgctatgcat gtgtcaacta atttaaacct aagcaatatt | 360 |
| ataaggaagg tactattata | |
| gaaatctcag ccttacaggt aagggaacag gaataaagag | 420 |
| atgtgaggta atggcccaag | |

For purposes of the present invention, this DNA sequence will be referred to as SEQ ID NO:5.

| | |
|---|---|
| ataatctcct ttcaagtttt tatcctgtca cttgctagtt | 60 |
| gtgtgatttg ggacaaatca | |
| tttaactcct tgtaaaggga gagaag [symbol] aag gctgtaaaaa | 120 |
| aattaagtaa taaaaagata | |
| aactccttgt ggtatatttt gttattgttc aaaaatattt | 180 |
| attgcccctc ttaggatgtc | |
| ttaggtcatt cttgcattgc tataaagaaa tacccaagtc | 240 |
| tgggtaattt ataaagaata | |
| gaggttaaat tggctcacag ttctgcaggc tgcacaggaa | 300 |
| gcatcccact ggcgtctact | |
| cacttctggt gaggactcag aaagcttttg cttatgacag | 360 |
| caggctaagt gagagcaggt | |

For purposes of the present invention, this DNA sequence will be referred to as SEQ ID NO:6.

Several additional sequences of DNA that are downstream from SEQ ID NO:3 are identified as relevant to the present invention. These DNA sequences are also found on NT_{—}022030 and are

| | |
|---|---|
| catggtattt ttactaccca ttgccttcta ggaaagggta | 60 |
| taacaaatag gaaatattaa | |
| tatttttaat gcctttgagg gtgttaaaaa gcacaactct | 120 |
| aaggactgtt tgtaaattc [symbol] | |
| aggtcaaatg ttgtttctcc ttctctattt cctaccttgg | 180 |
| tgatggcctg atcttatatg | |
| gagtcactcc aactagaaac cacagaatca tccctagttc | 240 |
| ctacttctga ctcactccat | |
| acactcaaaa gtcacctgac tctgcagaat ttctctagaa | 300 |
| aaactctatg aaaacctatt | |
| cctgcctctc cacctgcata gatgtagctt catccaggct | 360 |
| cttatggtgc atggcctcgg | |
| ttactgcctt atcctttcta ctggcctctc aatctcccat | 420 |
| ctgataccca ttaatgtact | |

For purposes of the present invention, this DNA sequence will be referred to as SEQ ID NO:7.

| | |
|---|---|
| ccaaatactt tttaggcaca ctgggaagtt acattgtttc | 60 |
| ttgcaagtga caggttgtcc | |
| tttaattagt tctttctctc aaaaagagac tgctgactcc | 120 |
| aaactgggaa gaaacccact | |
| caccagcaaa atgctgctga attcactctg atagttttct | 180 |
| aatctctcat cagtagatga | |
| caataatgaa gccagtattg ttaccacaag actcagatat | 240 |
| [symbol] tctatcacc caagatgatt | |
| tctctttaag acgcaataaa agggaacttt tctccccatt | 300 |
| tattagcaac taagatgaaa | |
| tgagagccag agaaataaag tgaggaagga aagagaattt | 360 |
| actaccttta caagctgaaa | |

For purposes of the present invention, this DNA sequence will be referred to as SEQ ID NO:8. In all upstream and downstream sequences (i.e. SEQ ID NOS: 4, 5, 6, 7, and 8), the location of SNPs are indicated by bold and larger font letters.

In Situ Hybridization

Double-stranded cDNA containing the RGS4 sequence was first amplified from normal human brain cDNA using custom designed primers (Forward primer sequence: CCGAAGCCACAGCTCCTC (SEQ ID NO: 3); Reverse primer sequence: CATCCCTCTCCCTTCAGGTG (SEQ ID NO: 4), and “touchdown” PCR with AmpliTaq Gold (PE Biosystems): (94° C. for 10 minutes (min), followed by 10 PCR cycles with a high annealing temperature 94° C. for 30 seconds (sec), 62° C. for 30 sec, and 72° C. for 60 sec), 10 cycles with a medium annealing temperature (94° C. for 30 sec, 60° C. for 30 sec, 72° C. for 60 sec), and 20 cycles at a low annealing temperature (94° C. for 30 sec, 58° C. for 30 sec, 72° C. for 60 sec). The product of this touchdown PCR reaction produced a single bright band on a 2% agarose gel and was purified and ligated into a T/A plasmid cloning vector (AdvanTAge, Clontech) and transformed into competent Escherichia coli cells and plated overnight at 37° C. Colony PCR was performed on selected colonies containing the insert, and the products of these reactions were restriction digested and sequenced to verify orientation and insert identity.

[³⁵S]-labeled riboprobes were synthesized using the T7 Riboprobe In Vitro Transcription System (Promega kit #P1460) and purified using RNeasy kit (Qiagen #74104). A scintillation counter was used to verify the specific radioactivity and yield of the probe. During hybridization, approximately 3 nanograms (ng) of probe was used per slide in a total volume of 90 μl. All other methods used were those described previously in Campbell et al., in Exp. Neurol. 160: 268-278, 1999, which is hereby incorporated by reference.

Tissue blocks containing the regions of interest (PFC area 9, motor cortex [MC] and visual cortex [VC]) were identified using surface landmarks and sulci (the superior frontal gyrus, the central sulcus and precentral gyrus, and the calcarine sulcus, respectively). After histological verification of the regions, 20 μm sections containing these regions were cut with a cryostat at −20° C., mounted onto gelatin-coated glass slides, and stored at −80° C. until use. The slides were coded so that the investigator performing the analysis was blind to the diagnosis of the subjects.

Following hybridization and washing, slides were air dried and exposed to BioMax MR film (Kodak) for 8-22 hours and then dipped in emulsion (NTB-2, Kodak), and exposed for 3-5 days at 4° C. High resolution scans of each film image were used for quantification of signal with Image (Scion Corporation, Fredrick, Md.), version 4.0b), and darkfield images were captured from the developed slides. Throughout all steps and procedures, subject pairs were processed in parallel. Hybridization of sections with sense RGS4 riboprobe, used as a specificity control, did not result in detectable signal.

Quantification was performed by subtracting the background white matter OD from the average signal OD measured in five non-overlapping rectangular regions on each section (3 sections per tissue block). In PFC and MC, these rectangular regions spanned cortical layers II-VI. Due to the lack of RGS4 signal in layer IV throughout the neocortex, and the great expansion of this layer in VC, the supragranular and infranular signal intensities were analyzed separately in VC. However, there were no significant differences in the levels of signal contained in the supra- and infragranular layers, so they were combined as a measure of overall VC signal intensity.

Each in situ hybridization was repeated three times in separate hybridization reactions. The resulting ODs were background-corrected and averaged. Visual cortex (V1) OD quantification, due to a bi-laminar transcript distribution, was performed separately for the supragranular and infragranular layers.

In order to search for novel candidate genes whose expression is consistently altered in schizophrenia, high-density cDNA microarrays (UniGEM-V, Incyte Genomics) were used to examine the expression patterns of over 7,800 genes and ESTs in post mortem samples of prefrontal cortex area 9 from six matched pairs of schizophrenic and control subjects.

Comparison and Statistical Analyses

As illustrated in FIG. 1B, a gene was determined to be expressed if the arrayed immobilized probe or target (the design of which is shown in FIG. 1A) was successfully amplified by PCR, produced a signal from at least 40% of the spot surface and had a signal/background ratio over 5-fold for either the cy3 or cy5 probe. Both images represent the same spot under cy3 and cy5 excitation, respectively. In this experiment, the balanced cy3 signal intensity (control or c-subject) was 6.2-fold brighter than the cy5 signal intensity (schizophrenic or s-subject).

Genes were comparably expressed between the control and experimental samples if the cy3/cy5 ratio or cy5/cy3 ratio was <1.6. Over 80% of observations fell into this class. Gene expression was changed between the two samples at the 95% confidence level (95% CL) if the cy3/cy5 or cy5/cy3 signal was 1.6-1.89. Gene expression was changed between the two samples at the 99% confidence level (99% CL) if the cy3/cy5 or cy5/cy3 signal was 1.9.

In the microarray analyses, data from experimental subjects were compared to data from matched control subjects in a pairwise design to control for the effects of age, race, sex and PMI on gene expression. To evaluate potential changes in gene group expression on the microarrays, two types of statistical measures were employed: 1) χ-square analysis was performed on the distribution of genes in a group versus the distribution of all genes called present on each individual microarray. The distribution of gene expression ratios was divided into five different bins based on confidence levels for individual gene comparisons: <−1.9, −1.89 to −1.6, −1.59 to 1.59, 1.6 to 1.89 and >1.9. 2) A paired t-test (degrees of freedom=5) was used to compare mean expression ratios for a given gene group to the mean expression ratios for all expressed genes across all six subject pairs. A gene group was considered to be changed only if it reported differential expression by both the χ-square and t-test compared to the mean and distribution of all expressed genes. Microarray changes were also analyzed by descriptive statistics and correlation.

To mimic the microarray comparisons, the in situ hybridization data were analyzed using ANCOVA with diagnosis as the main effect, subject pair as a blocking factor, and brain pH and tissue storage time as covariates. Furthermore, to verify that the pairing of subjects adequately controlled for sex, age, and PMI, we also conducted an ANCOVA with diagnosis as a main effect, and sex, age, PMI brain pH, and tissue storage time as covariates. Since both models produced similar results, the values from the ANCOVA with subject pair as a blocking factor are reported. Changes between groups were also analyzed by descriptive statistics, Pearson correlation, and Factor analysis.

Pittsburgh Cases and Parents for Genotyping Analysis

Inpatients and outpatients were recruited at Western Psychiatric Institute and Clinic, a University of Pittsburgh-affiliated tertiary care center and 35 other treatment facilities within a 500 mile radius of Pittsburgh. The Diagnostic Interview for Genetic Studies (DIGS) was the primary source for clinical information for probands (Nurnberger, et al. Archives of General Psych. 51, 849-59; discussion 863-4, 1994). Additional information was obtained from available medical records and appropriate relatives, who also provided written informed consent. Consensus diagnoses were established by board certified psychiatrists. There were 93 Caucasian and 70 African-American cases. Genomic DNA, but not clinical information was available from all parents of the Caucasian cases. Cord blood samples were obtained from live births at Pittsburgh and served as unscreened, population-based controls. There were 169 individuals. They included 76 Caucasians and 93 African-Americans.

National Institute of Mental Health Collaborative Genetics Initiative (NIMH CGI) Sample

From 1991-98, pedigrees having probands with schizophrenia or schizoaffective disorder, depressed (DSM IV criteria) were ascertained at Columbia University, Harvard University, and Washington University. The DIGS was the primary interview schedule. The families were ascertained if they included two or more affected first degree relatives (Cloninger et al. Am. J. Med. Gen. 81, 275-81, 1998, which is hereby incorporated by reference). We selected case-parent trios and available affected siblings from this cohort. Thus, 39 cases, their parents and 30 affected sibling-pairs were obtained. They comprised 25 Caucasian families, 10 who reported African-American ethnicity and 4 from other ethnic groups. Transmission disequilibrium test (TDT) analysis utilized only one case/family.

Written, informed consent was obtained from all participants. Ethnicity was based on self-report (maternal report for neonatal samples).

DNA Sequencing and Polymorphism Detection

The genomic sequence for RGS4 was obtained from NT_{—}022030 (390242 bp), a currently unfinished clone from Human Genome Project, Chromosome 1 database. The annotated data revealed three identified genes, namely, RGS4, MSTP032 and RGS5. The genomic organization of RGS4 and RGS5 includes 5 exons which is typical for the RGS family gene.

A panel of 10 African-American cases and 6 Caucasian controls was initially used to screen for polymorphisms in the exonic, intronic, and flanking genomic sequences of the RGS4 gene. The re-sequenced region included 6.8 kb upstream and 2.9 kb downstream of the coding sequence. The genomic sequence was used to design primers and amplicons 500 bp were generated, with overlapping sequences. The amplified fragments were sequenced using an ABI 3700 DNA sequencer. The sequencing panel that was used (n=16) has over 80% power to detect SNPs with minor allele frequency over 5% (Kruglyak et al. Nature Gen. 27, 234-236, 2001, which is hereby incorporated by reference). We also sequenced cDNA sequences from the post-mortem samples reported on earlier (Mirnics et al. Mol. Psychiatry 6, 293-301, 2001). The sequences were aligned using Sequencher (version 4.5) and polymorphisms were numbered consecutively. Additional SNPs localized to NT_{—}022030 were obtained from the NCBI SNP database (“http://www.ncbi.nlm.nih.gov/SNP”). We also obtained genotype data from a prior study of the NIMH sample (“http://zork.wustl.edu/nimh”).

Polymorphism Analysis

PCR based assays included primers (5 pmol) with 200 μM dNTP, 1.5 mM MgCl2, 0.5 U of AmpliTaq Polymerase (PE Biosystems), 1× buffer and 60 ng of genomic DNA in 10 or 20 μl reactions. The PCR conditions were 95° C. for 10 min followed by 35 cycles (94° C. for 45 sec, 60° C. 45 sec and 72° C. for 1 min). The final extension at 72° C. for 7 min. The amplified products were digested with restriction endonucleases, electrophoresed on agarose gels, and visualized using ethidium stain. SNPs 4 and 18 were identified as single strand conformational polymorphisms (SSCP) (Orita et al. DNAS 86, 2766-70, 1989). All genotypes were read independently by two investigators.

Polymorphisms were detected only in the intronic and flanking sequences of RGS4 (FIG. 6). Among 34 identified SNPs, one was selected from each of six sets which appeared to be in complete linkage disequilibirum in the re-sequenced panel. SNPs were further evaluated for informativeness (minor allele frequency >0.1) and availability of reliable genotyping assays. Among the Caucasian cases from Pittsburgh, deviations from Hardy Weinberg equilibrium (HWE) were noted for SNP 7 (p<0.03) and SNP 13 (p<0.01). Though all maternal genotypes conformed to HWE, deviations were noted at SNPs for the fathers of Pittsburgh cases at SNPs 4 and 18 (p<0.05). For the analysis of IBD sharing among affected sibling-pairs from the NIMH samples, we also used genotypes for markers D1S1595, D1S484, D1S1677, D1S431 and D1S1589 (Faraone et al. Am. J. of Med. Gen. 81, 290-5, 1998).

Statistical Analysis

PEDCHECK software was used to check for Mendelian inconsistencies (O'Connell et al. Am. J. of Hum. Gen. 63, 259-266, 1998, which is hereby incorporated by reference). χ² tests were employed for comparisons between cases and unrelated controls. We also used SNPEM software based on the EM algorithm to estimate and compare haplotype frequencies (Fallin, 2001, which is hereby incorporated by reference). We utilized GENEHUNTER software for TDT analysis of individual SNPs and haplotypes, as well as analysis of identity by descent among affected sibling-pairs (Kruglyak et al. Am. J. of Hum. Gen. 58, 1347-63, 1996; Spielman et al. Am. J. of Hum. Gen. 54, 559-60, 1994, both of which are hereby incorporated by reference). We also used TRANSMIT for global tests of association involving multiple haplotypes (Clayton et al. Am. J. of Med. Gen. 65, 1161-1169, 1999a; Clayton et al. Am. J. of Hum. Gen. 65, 1170-1177, 1999b, both of which are hereby incorporated by reference).

Microarray Results

Single gene transcripts were analyzed across all cDNA microarray comparisons. Across the six microarray comparisons over 90,000 data points were collected, and from these 44,000 were expression-positive observations, resulting in an average of 3,735 expressed genes/microarray. Of the expressed transcripts, 4.8% were judged to be differentially expressed (99% CL) between the schizophrenic and control subjects. The observed differences for any subject pair, in general, were comparably distributed in both directions: 2.6% of the genes were expressed at higher levels in schizophrenic subjects than in the matched controls, whereas 2.2% were expressed at lower levels in the schizophrenic subject.

Of all the expressed genes, RGS4 transcript reported the most significant decrease across all schizophrenic subjects. In fact, it was the only gene decreased at the 99% CL in all microarray comparisons. The microarray-bound, 571 base pair long, double-stranded cDNA immobilized probe corresponded to the 3′ end of RGS4 and had a less than 50% sequence homology to any other known transcript, including RGS family members. This high binding specificity, coupled with strong cy3 and cy5 hybridization signal intensities, as shown in FIG. 1B, showed that RGS4 was robustly expressed in the human prefrontal cortex. Across the six microarray comparisons, RGS4 mRNA levels were decreased 50-84% in the PFC of schizophrenic subjects, as illustrated in FIG. 1C, while the expression of the ten other RGS family members represented on the microarray were unchanged in the schizophrenic subjects. In the scatter plot shown in FIG. 1C, the X-axis reports subject pairs, the Y-axis reports percent change between schizophrenic and control subjects. Individual symbols represent a gene expression difference between a schizophrenic and control subject in a single pairwise comparison. The black dashed line denotes equal cy3 and cy5 signal intensity (similar expression) between schizophrenic and control subjects (0% change), green dashed line denotes the 95% confidence interval (37.5% change), red dashed line represents 99% confidence interval (47.5% change). Missing symbols in some pairwise comparisons indicate that the corresponding genes' microarray hybridization did not meet expression criteria. Across all the RGS members represented on the microarray, only RGS4 showed a consistent expression change over the 99% CL in schizophrenic subjects.

To confirm the microarray findings for the RGS4 expression changes, in situ hybridization was performed on the PFC from the same five subject pairs used for the microarray experiments (for pair 794c/665s, no sections were available from the same block of tissue used in the microarray experiment). As a further test of the robustness of the microarray data, five additional subject pairs were added to the in situ hybridization analysis. Radiolabeled cRNA probes designed against RGS4 mRNA were used to localize and quantify relative transcript levels. In the control subjects, RGS4 labeling was heavy in the prefrontal cortex, as shown in FIG. 2A, mimicking previously described labeling in the rat. In the gray matter of prefrontal cortex, the RGS4 riboprobe heavily labeled various size and shape cell profiles, including both projection neurons and interneurons. This labeling was the most prominent in layers III and V, with sparse labeling in the intervening granular layer IV, and appeared to be present over both large pyramidal neurons and smaller cells that could represent interneurons. High power photomicrographs of PFC tissue sections from a schizophrenic (622s) and matched control (685c) subjects were viewed under darkfield illumination. Micrographs for each subject were taken under identical conditions. Roman numbers denote cortical layers. Pial surface is to the left. Strong labeling across all cortical layers except lamina IV was observed, and diminished labeling in the matched schizophrenic subject across all the layers was noted (scale bar=400 μm). White matter labeling was absent.

Based on optical density analysis, 9/10 subject pairs exhibited a 10.2% to 74.3% decrease in PFC RGS4 expression, as shown in FIG. 2B. The in situ hybridization data from 10 PFC pairwise comparisons were quantified using film densitometry. The X-axis represents subject classes, the Y-axis reports average film OD from 3 repeated hybridizations, measured across all layers. Lines connecting symbols indicate a matched subject pair. Note that in 10 PFC pairwise comparisons, 9 schizophrenic subjects showed RGS4 transcript reduction (mean=−34.5%; F_{1,1}₅=6.95; p=0.019).

Specificity of RGS4 Expression Changes

To investigate whether RGS4 transcript decrease is a specific alteration in schizophrenia, the same microarray data was analyzed for consistent gene expression changes across other RGS-family members (FIG. 1C). Nine of the eleven RGS family members represented with immobilized probes on the microarrays reported expression in four or more microarray comparisons. RGS13, primarily lung-specific family member, was not expressed in any of the comparisons, while p115-RhoGEF reported expression in only one comparison. RGS4 was the only family member (and the only gene on the microarray) to report a consistent change in expression over the 99% CL in every schizophrenic subject. RGS5 mRNA (a gene also localized to cytogenetic position 1q21-22) was decreased at the 99% CL in one subject pair, at the 95% CL in another subject pair, and unchanged in the remaining 2 pairs that showed detectable RGS5 expression by microarrays. Expression of the other RGS family members did not display any consistent differences across the schizophrenic subjects. The mRNA from pair 567c/537s was analyzed a second time on the newest Incyte microarray, UniGEM-V2, which includes five additional RGS family members (RGSZ, RGS1, RGS7, RGS11, and RGS14). This analysis confirmed that, in the comparisons, RGS4 was the only significantly changed RGS family member.

Heterotrimeric G-proteins, the main substrates for RGS family members, were assessed for expression patterns. Several reports suggest Gα changes associated with schizophrenia. Thus, it was desirable to assess whether the decrease in RGS4 expression correlated with changes in Gα expression levels. Of the eight Gα RGS substrates represented on the microarrays, only Gₒ expression was changed beyond the 95% CL in three or more pairwise comparisons. These three subjects with increased Go levels (317s, 547s, and 622s) showed the most robust decrease in RGS4 expression both in the PFC microarray and in situ hybridization assays.

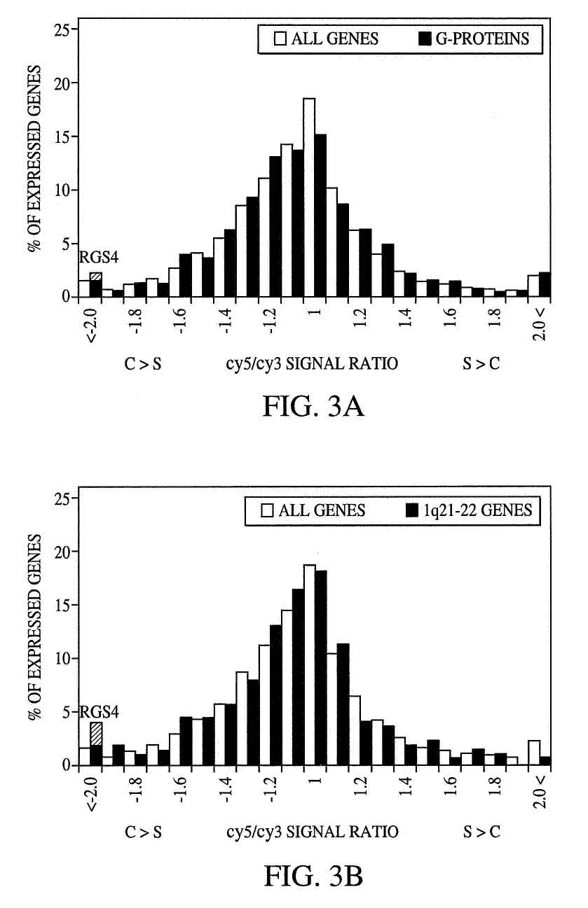

Expression of 274 genes known to be involved in the G-protein signaling cascades (GPCR, heterotrimeric G-proteins, RGS, GIRKs, G-protein receptor kinases, and mitogen-activated protein kinases) were analyzed in a gene group comparison. An average of 105 genes belonging to this group were expressed in each comparison. The results of microarray analyses showing G-protein and 1q21-22 locus-related expression differences in the PFC of six pairs of schizophrenic and control subjects are shown in FIGS. 3A and 3B. For both gene groups, all expressed genes were classified into signal intensity difference intervals (0.1 bins) according to their cy5/cy3 signal ratio. Transcripts in a “1” bin had identical cy5 vs. cy3 signal intensities. Positive values (to the right) on the X-axis denote higher cy5 signal in schizophrenic subjects (S>C), negative values (to the left) correspond to higher cy3 signal intensity in the control subjects (C>S). The Y-axis reports percentage of expressed genes across the six subject pairs per bin for each gene group. In both panels, the white bars (All genes) denote distribution of all expressed genes across the six PFC pairwise comparisons (n=22,408). Additionally, in both panels, RGS4 contribution to the transcript distribution is denoted by a hatched bar. Note that in both FIG. 3A and FIG. 3B, the cy3/cy5 signal distribution of G-protein and 1q21-22 gene groups was comparable to the distribution of all expressed genes across the six microarray comparisons.

At the 99% confidence level, 5.6W of G-proteins showed a different distribution between schizophrenic and control subjects, as shown in FIG. 3A: 2.8% of G-proteins were decreased, while 2.8% were increased in the PFC of schizophrenic subjects. Of the 2.8% decrease in schizophrenic subjects, RGS4 observations alone accounted for nearly half of the decrease. When RGS4 was removed from the G-protein group, a gene group analysis by χ² test and t-test closely matched the distribution of all expressed genes, suggesting that the majority of different expression levels can be attributed to normal human variability. Except RGS4, no other member of the G-protein gene group was consistently changed across the subject pairs over the 95% or 99% confidence levels.

The RGS4 gene has been mapped to locus 1q21-22, a novel schizophrenia locus recently implicated by pedigree studies with a linkage of disease score (LOD) of 6.5 as described by Brzustowicz et al. supra. To address if any other genes at this locus displayed altered expression in the PFC of schizophrenic subjects, 70 additional transcripts originating from this cytogenetic region were analyzed. At the 99% CL, 0.4% of 1q21-22 genes were increased, and 5.9% were decreased in the schizophrenic subjects. Of the transcripts decreased in schizophrenic subjects, RGS4 observations alone accounted for nearly half of the decreases, as shown in FIG. 3B. Furthermore, of all the genes on the 1q21-22 locus, only RGS4 showed a consistent expression change across all the pairwise comparisons over the 95% or 99% confidence levels. Of the remaining genes on this locus, only the ALL1-FUSED gene (AF1q GenBank Accesion #U16954) reported consistent expression change over the 95% CL in the schizophrenic subjects in three or more pairwise comparisons. Furthermore, as a gene group, the expression of the remaining genes on locus 1q21-22 showed the same overall pattern as genes located on non-schizophrenia loci or the overall average gene expression which is shown in FIG. 3B.

Regional RGS4 Gene Expression Changes

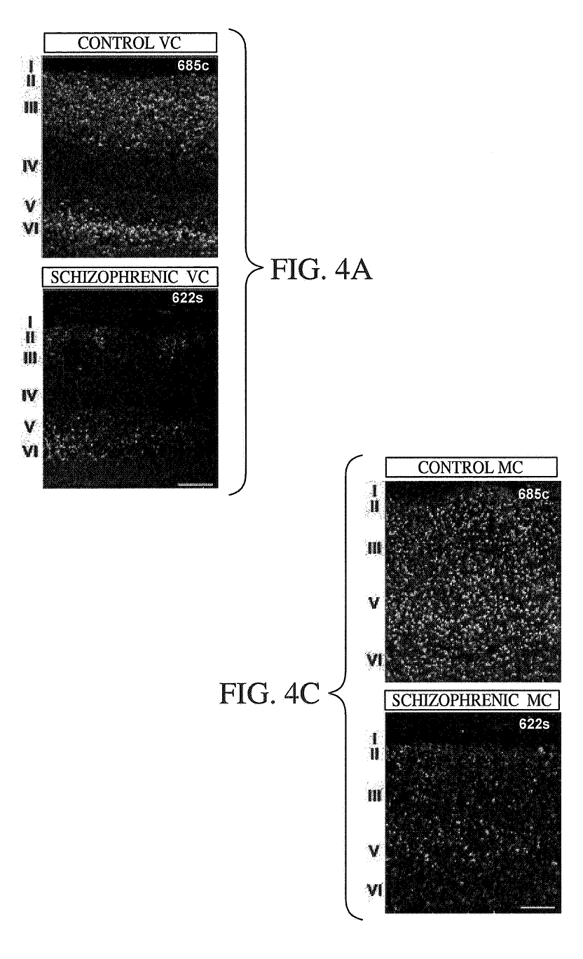

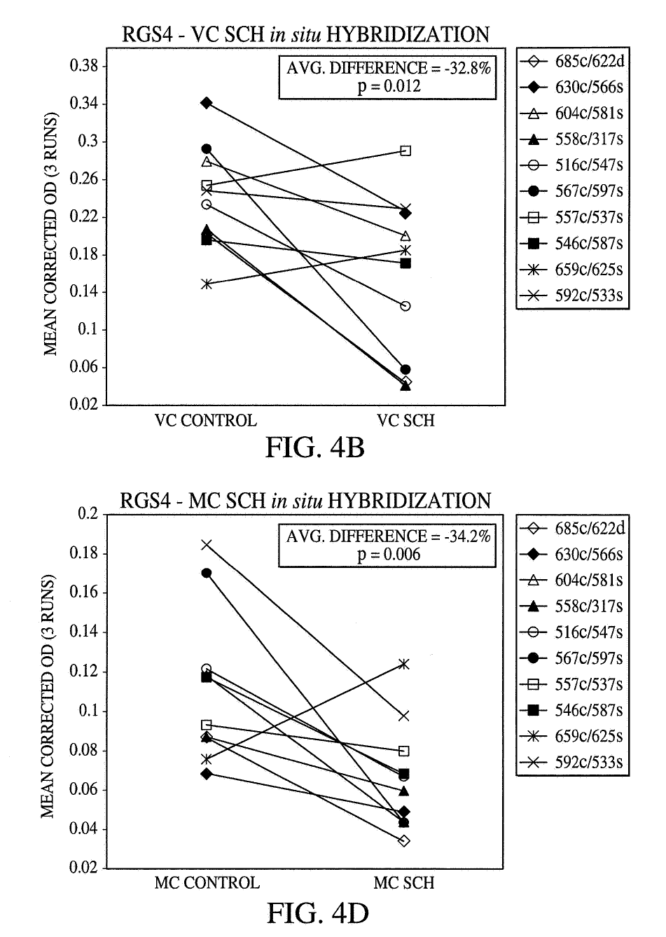

To test whether RGS4 transcript decrease is specific to the prefrontal cortex or includes a more widespread cortical deficiency, RGS4 expression was assessed by in situ hybridization in the visual cortex (VC) and motor cortex (MC) from the same 10 pairs of control and schizophrenic subjects (for pair 558c/317s MC material was not available, and this pair was substituted with pair 794c/665s). The figure layout for FIG. 4A-D is similar to that of FIG. 2A-B. In VC, RGS4 in situ hybridization showed heavy labeling under darkfield illumination of diverse cell population in the gray matter, with a very prominent bi-laminar labeling pattern in the supragranular and infragranular layers, as shown in FIG. 4A. Roman numbers denote cortical layers, scale bar=400 μm. There was very sparse labeling in the well-developed layer IV, with very few cellular elements exhibiting detectable levels of RGS4 mRNA. These high power photomicrographs show that RGS4 levels are significantly decreased in the VC region of the schizophrenic subjects. The OD measurements on these two layers were performed separately.

Across the same ten pairwise comparisons that were examined in the PFC hybridizations, combined RGS4 expression in supragranular and infragranular layers of VC was decreased by 32.8% (F_{1,15}=8.24; p=0.012) as shown in FIG. 4B.

In MC, RGS4 expression was concentrated over the cell-rich layers I-III and V-VI of both control and schizophrenic subjects, as shown in FIG. 4C. High power photomicrographs of MC tissue sections from the same matched pair of schizophrenic and control subject are represented in FIG. 2A and FIG. 4A, viewed under darkf ield illumination. Roman numbers denote cortical layers, scale bar=400 μm. Because of the attenuated layer IV in motor cortex, the RGS4 labeling is almost uniform across all layers.

Similar to the RGS4 transcript decrease observed in supragranular VC, schizophrenic subjects across the same 10 subject pairs were analyzed in MC. The mean RGS4 expression in MC shown in FIG. 4D, measured across all the layers, was decreased by 34.2% across the 10 schizophrenic subjects (F_{1,15}=10.18; p=0.006).

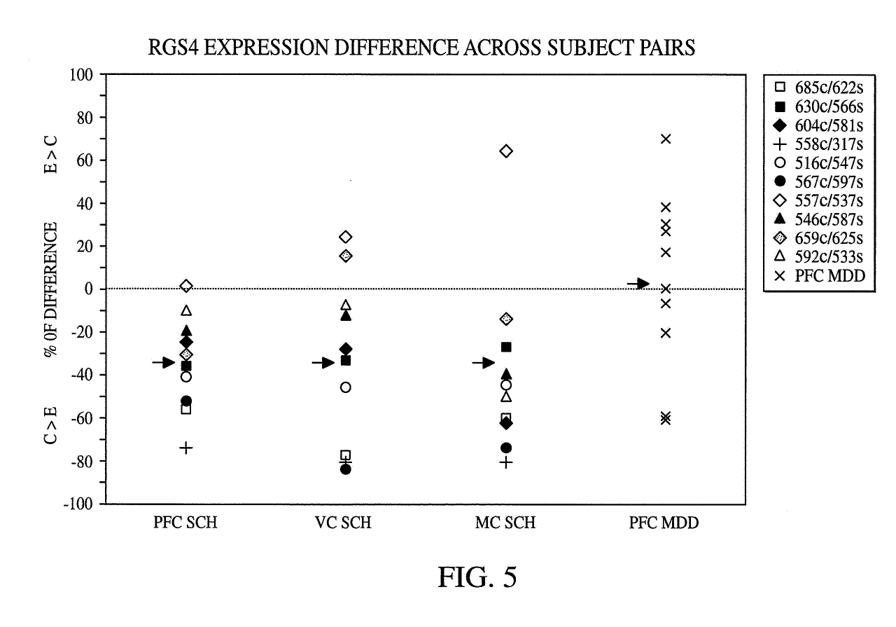

In the PFC, VC, and MC of subjects with schizophrenia, RGS4 expression was consistently decreased compared to the PFC of subjects with the diagnosis of MDD, as shown in the schematic of FIG. 5. In contrast, factor analysis of the pairwise differences in RGS4 gene expression across 3 different cortical areas for all 9 common schizophrenic and control subject pairs revealed that over 84% of the total variance in expression was accounted for by diagnosis (variance proportion=0.848, eigenvalue=2.544, p=0.001. The X-axis represents experimental groups, the Y-axis reports percent RGS4 expression change in PFC, VC, MC, in schizophrenic subjects (SCH) and PFC of subjects with MDD viewed by in situ hybridization. Each symbol represents percent of change between a single pairwise comparison; same symbols represent the same subject pairs. Arrows represent mean expression difference for each group. The same schizophrenic subjects showed a comparable and highly correlated decrease in RGS4 expression across all three cortical regions (PFC-VC: r=0.88, p=0.0003; PFC-MC: r=0.69, p=0.0384; VC-MC: r=0.76, p=0.0144). In contrast, subjects with MDD reported variable RGS4 expression changes when compared to their matched controls.

The combined data indicate that RGS4 transcript changes are a result of the pathophysiological changes related to schizophrenia and not due to confounds. Furthermore, the RGS4 expression decrease appears to be specific and unique to schizophrenia, and not a hallmark of the major depressive disorder.

RGS4 labeling in the white matter was comparable to background labeling across all brain regions, suggesting that RGS4 is primarily expressed in neuronal cells. The labeling was abundant in the majority of interneurons and projection neurons. However, in some pyramidal cells and interneurons RGS4 labeling could not be detected. RGS4 labeling was heavy in all cortical layers, except layer IV, where RGS4 expression was both sparse and light. This overall pattern of labeling was comparable across all three cortical regions (PFC, VC, MC). As the granular layer IV is the widest in the primary visual cortex, in this region RGS4 labeling was prominent in supragranular and infragranular layers, separated by a wide zone of mostly unlabeled granular cells. The overall distribution pattern of the RGS4 message does not mimic the known expression patterns of neurotransmitter systems, suggesting that RGS4 regulates many functionally distinct neuronal populations.

Together, the microarray and in situ hybridization methods suggest decreased RGS4 expression is a consistent characteristic of schizophrenic subjects. Several causes of the reduced RGS4 expression may be offered, including adaptive and genetic changes in schizophrenic patients. It was hypothesized that reduction in RGS4 expression was generated by alterations in the RGS4 gene. In addition, it was contemplated that variations in the DNA upstream and downstream from the coding region of the RGS4 gene may also impact the expression of the RGS4 transcript. These possibilities were investigated by searching for SNPs in the RGS4 gene.

The specificity of the reduced expression of RGS4 message for schizophrenic patients was confirmed in a series of control experiments. The same reduced level of RGS4 message was not observed in patients suffering from major depressive disorder. In addition, prolonged treatment of non-human primates with the anti-psychotic haloperidol did not result in decreased levels of RGS message in the cerebral cortex. This result indicates that chronic exposure to anti-psychotic drugs are unlikely to be responsible for the depressed levels of RGS4 message observed in schizophrenic patients.

Genotyping Results

34 single nucleotide polymorphisms (SNPs) were identified after re-sequencing all exons, introns and flanking 5′ and 3′ UTRs of the RGS4 coding region (FIG. 6). Thirteen SNPs were chosen for analysis using the TDT. SNPs are explicitly defined in Table 1. When the SNPs were tested individually, significantly increased transmission at SNP4 was observed in the Pittsburgh sample. ‘Moving window’ haplotype analyses using two to four contiguous SNPs, revealed significant association for several haplotypes; all but one included SNPs 1, 4, 7, or 18 (Table 2). A global test of association for haplotypes encompassing these SNPs was significant (TRANSMIT software, χ²=16.6, 8 df, p=0.035). There were 39 cases with schizoaffective disorder in the sample; these trends remained significant when the sample was restricted to individuals with schizophrenia (χ²=13.0, 6 df, p=0.043).

TDT analysis was conducted next in the ethnically diverse NIMH sample using the same set of SNPs. Significant transmission distortion was observed individually at SNPs 1, 4 and 18 (Table 2). Exclusion of African-American families from the sample also

TABLE 1

Location of single nucleotide polymorphisms
relevant to the present invention. The location
of the SNP within the sequence is listed as is
the variation observed in the collected sam-
ples. SNP 14 is the absence of the listed 7
bases at the indicated location.

| | | | Observed |
|---|---|---|---|
| | | Nucleotide | Nucleo- |
| | Location of the SNP | identity in | tide |
| SNP # | within the SEQ | SEQ ID NO: 3 | variation |
| 27,859 | 199 {SEQ ID NO: 4} | T | C |
| 34,653 | 153 {SEQ ID NO: 5} | C | T |
| 90,387 | 87 {SEQ ID NO: 6} | G | A |
| SNP1 | 4121 {SEQ ID NO: 3} | C | T |
| SNP2 | 4123 {SEQ ID NO: 3} | T | A |
| SNP3 | 4368 {SEQ ID NO: 3} | A | C |
| SNP4 | 4621 {SEQ ID NO: 3} | A | C |
| SNP5 | 4790 {SEQ ID NO: 3} | C | T |
| SNP6 | 4816 {SEQ ID NO: 3} | G | T |
| SNP7 | 4970 {SEQ ID NO: 3} | C | T |
| SNP8 | 5055 {SEQ ID NO: 3} | C | G |
| SNP9 | 5295 {SEQ ID NO: 3} | G | A |
| SNP10 | 5695 {SEQ ID NO: 3} | G | A |
| SNP11 | 7375 {SEQ ID NO: 3} | G | T |
| SNP12 | 7759 {SEQ ID NO: 3} | G | A |
| SNP13 | 8596 {SEQ ID NO: 3} | G | A |
| SNP14 | 9603-9609 | AGTTTGG | 7 bases |
| | {SEQ ID NO: 3} | | Absent |
| SNP15 | 9892 {SEQ ID NO: 3} | C | A |
| SNP16 | 9963 {SEQ ID NO: 3} | C | A |
| SNP17 | 10132 {SEQ ID NO: 3} | G | A |
| SNP18 | 11056 {SEQ ID NO: 3} | T | C |
| SNP19 | 11091 {SEQ ID NO: 3} | C | T |
| SNP20 | 11106 {SEQ ID NO: 3} | C | A |
| SNP21 | 11774 {SEQ ID NO: 3} | G | T |
| SNP22 | 12143 {SEQ ID NO: 3} | G | A |
| SNP23 | 12145 {SEQ ID NO: 3} | G | T |
| SNP24 | 14367 {SEQ ID NO: 3} | A | G |
| SNP25 | 17028 {SEQ ID NO: 3} | A | Base |
| | | | absent |
| SNP26 | 17630 {SEQ ID NO: 3} | G | T |
| 118740 | 120 {SEQ ID NO: 7} | C | G |
| 130121 | 221 {SEQ ID NO: 8} | G | C |

revealed significant results for these SNPs (p=0.023, 0.011 and 0.033 respectively). However, the transmitted alleles differed from the Pittsburgh sample. Moving window haplotype analyses revealed preferential transmission for more extensive chromosomal segments than the Pittsburgh sample. Like the Pittsburgh sample, all but one of haplotypes with significant increased transmission included SNPs 1, 4, 7 or 18. A global test for association was also significant for haplotypes encompassing these SNPs (TRANSMIT analysis; χ²=18.8, p=0.016, 8 df).

If the significant TDT results were due to linkage, it was reasoned that the affected sibships in the NIMH sample should yield evidence for increased haplotype sharing. For 30 available affected sib-pairs, the proportion of 0, 1, or 2 haplotypes identical by descent (IBD). were elevated over expectations of 0.25, 0.50, 0.25; namely 0.11, 0.44, 0.45 respectively (for SNPs 1, 4, 7 and 18 analyzed in conjunction with 5 flanking short tandem repeat polymorphisms genotyped previously). Increased IBD sharing was also observed when these sets of SNPs or STRPs were analyzed separately.

Association at the population level was assessed by comparing Caucasian cases from each sample separately with two independent groups of Caucasian community-based controls. Since SNPs 1, 4, 7 and 18 appeared to be critical for transmission distortion in both samples, genotypes and allele frequencies for these SNPs were analyzed. Haplotypes frequencies were estimated using an expectation-maximization algorithm (EM), paying particular attention to haplotypes VI and XI, the haplotypes with excess transmission in the NIMH and tsburgh samples, respectively (Table 3). SNP 14 was ormative only among African-Americans and so was lyzed separately using 70 African-American cases and control individuals from Pittsburgh. Significant e-control differences were not noted for any of the parisons. The failure to detect association may lect superior power for the TDT in the context of ulation sub-structure.

TABLE 2

| | | Neonatal | Adult | Pittsburgh | |
|---|---|---|---|---|---|
| No. | Haplotype | Controls | controls | Cases | NIMH Cases |
| | SNP | | | | |
| | 1-4-7-18 | | | | |
| I | 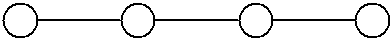 | 0.096 | 0.066 | 0.078 | 0.067 |
| II | 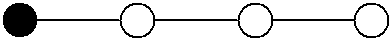 | 0.004 | 0.021 | 0.022 | 0.083 |
| III | 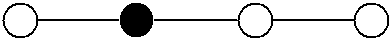 | 0.006 | 0.006 | 0.000 | 0.000 |
| IV | 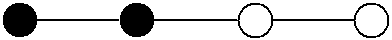 | 0.000 | 0.000 | 0.000 | 0.000 |
| V | 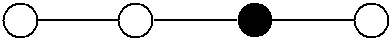 | 0.000 | 0.000 | 0.006 | 0.000 |
| 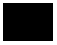 | 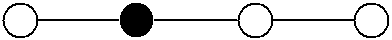 | 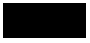 | 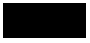 | 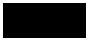 | 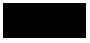 |
| VII | 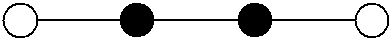 | 0.000 | 0.006 | 0.000 | 0.000 |
| VIII | 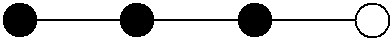 | 0.000 | 0.000 | 0.006 | 0.000 |
| IX | 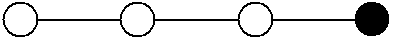 | 0.000 | 0.004 | 0.000 | 0.017 |
| X | 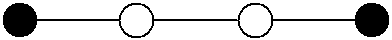 | 0.000 | 0.006 | 0.000 | 0.000 |
| 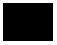 | 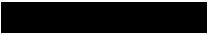 | 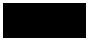 | 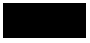 | 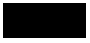 | 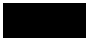 |
| XII | 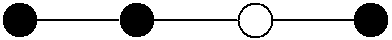 | 0.008 | 0.013 | 0.000 | 0.000 |
| XIII | 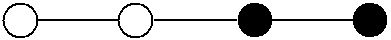 | 0.000 | 0.000 | 0.000 | 0.000 |
| XIV | 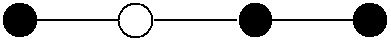 | 0.053 | 0.013 | 0.017 | 0.025 |
| XV | 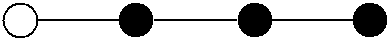 | 0.006 | 0.000 | 0.000 | 0.000 |
| XVI | 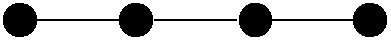 | 0.000 | 0.000 | 0.000 | 0.000 |

Haplotype based comparisons among cases and
unrelated controls. The Caucasian cases from Pittsburgh (n = 93) and
NIMH (n = 25) were compared separately with unscreened Caucasian
controls from Pittsburgh (n = 76). Bonferoni corrections have been
applied for the Pittsburgh case-control comparisons, but not for
comparisons involving the NIMH cases. An omnibus test based on likelihood
ratios was used to estimate overall differences in haplotype frequencies
(Fallin et al., Gen. Res. 11, 143-51, 2001) and was significant for
both comparisons (χ² = 88.7, p < 0.0001 and
χ² = 30.1, p < 0.0003 respectively for Pittsburgh and
NIMH cases). Similar significant differences based on 3 SNP haplotypes
were present, but are not shown. For each SNP

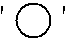

represents allele 1 and

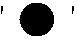

represents allele
2. OR—Odds ratio; NS—Not significant.

TABLE 3
Pair-wise linkage disequlibrium between SNPs
at RGS4. Population based control individuals (n = 76)
were used to estimate linkage disequilibrium. The
figures above the diagonal represent D′ and estimates
for statistical significance (p values) are below the
diagonal.

| SNP | 27859 | 90387 | snp1 | snp4 | snp7 | snp18 | snp23 | 118740 | 130121 |
|---|---|---|---|---|---|---|---|---|---|
| 27859 | | 0.096 | 0.064 | 0.076 | 0.287 | 0.009 | 0.000 | 0.000 | 0.000 |
| 90387 | 0.132 | | 0.000 | 0.000 | 0.000 | 0.000 | 0.000 | 0.001 | 0.627 |
| snp1 | −0.123 | −0.501 | | 0.000 | 0.000 | 0.000 | 0.000 | 0.450 | 0.477 |
| snp4 | 0.101 | −0.501 | −1.000 | | 0.000 | 0.000 | 0.000 | 0.128 | 0.515 |
| snp7 | −0.075 | 0.783 | 0.970 | −0.961 | | 0.000 | 0.000 | 0.012 | 0.068 |
| snp18 | 0.177 | 0.377 | −0.677 | 0.989 | −0.961 | | 0.000 | 0.000 | 0.041 |
| snp23 | 0.527 | −0.302 | −1.000 | 1.000 | −0.847 | 0.674 | | 0.499 | 0.002 |
| 118740 | 0.385 | 0.163 | 0.048 | −0.083 | 0.172 | −0.233 | 0.042 | | 0.000 |
| 130121 | −0.505 | 0.049 | −0.059 | 0.046 | −0.163 | 0.174 | −0.154 | −0.956 | |

TABLE 4

| | |
|---|---|
| Pittsburgh | NIMH |

| | | | | | |
|---|---|---|---|---|---|
| SNP | Transmitted allele | T/NT | SNP | Transmitted allele | T/NT |
| SNP 1 | 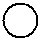 | 53/35 (0.055) | SNP 1 | 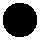 | 30/13 (0.01) |
| SNP 4 | 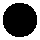 | 51/33 (<0.05) | SNP 4 | 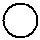 | 22/6 (0.003) |
| | | | SNP 18 | 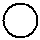 | 24/8 (0.005) |

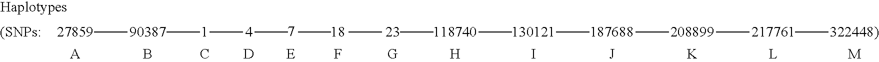

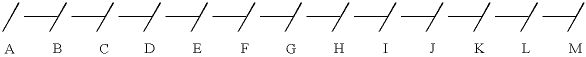

| | |
|---|---|
| 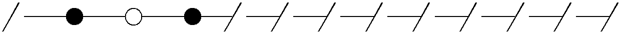 | 34/18(0.03) |
| 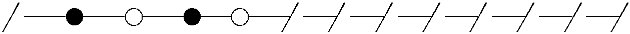 | 33/14(0.006) |
| 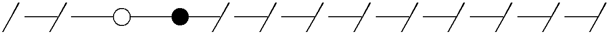 | 38/17(0.005) |
| 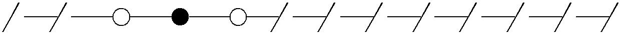 | 37/13(0.0007) |
| 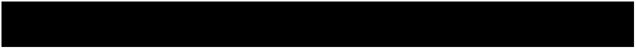 | 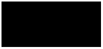 |
| 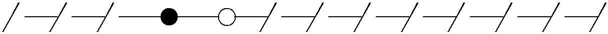 | 39/19(0.009) |
| 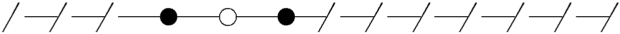 | 39/19(0.009) |
| 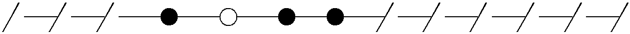 | 35/19(0.03) |
| 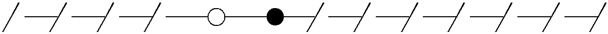 | 40/22(0.022) |
| 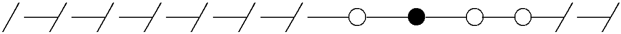 | 6/0(0.01) |

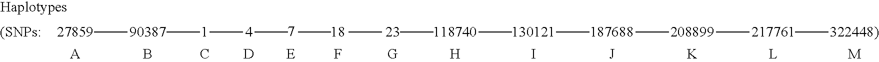

| 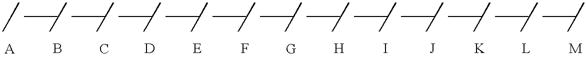 | |
|---|---|
| 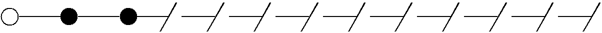 | 9/1 (0.02) |
| 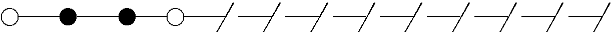 | 8/0 (0.005) |
| 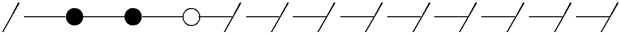 | 11/3 (0.04) |
| 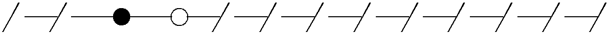 | 23/3 (0.0001) |
| 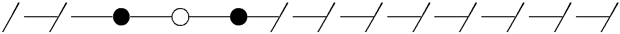 | 19/3 (0.001) |
| 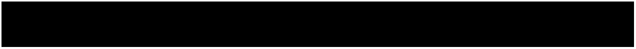 | 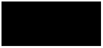 |
| 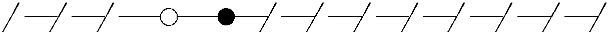 | 20/7 (0.02) |
| 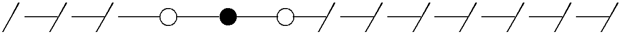 | 20/7 (0.02) |
| 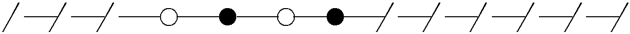 | 11/3 (0.04) |
| 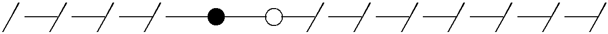 | 20/7 (0.02) |
| 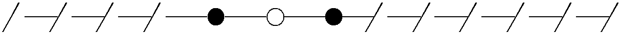 | 11/3 (0.04) |
| 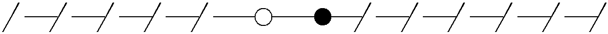 | 20/6 (0.006) |
| 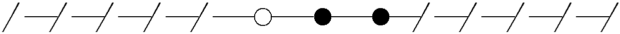 | 20/4 (0.001) |
| 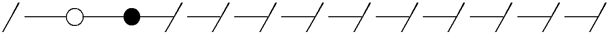 | 11/3 (0.04) |
| 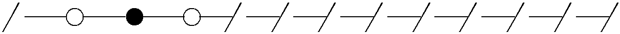 | 11/3 (0.04) |
| 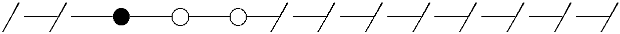 | 4/0 (0.05) |
| 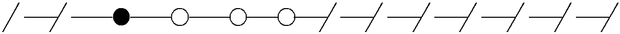 | 4/0 (0.05) |
| 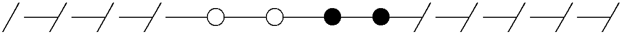 | 7/1 (0.04) |
| 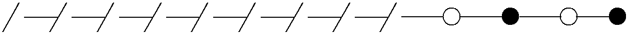 | 5/0 (0.03) |

SNPs and Haplotypes at RGS4 with increased
transmission distortion. TDT analysis of case-parent trios
included 93 families from Pittsburgh and 39 families from the
NIMH cohort. Only statistically significant increased
transmissions are shown. The shaded haplotypes correspond to
haplotypes VII and X, respectively from Table 2. T/NT-
Transmitted/not transmitted;

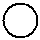

Allele 1,

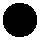

Allele 2 at each SNP;/-Allele not specified at this locus;
*p < 0.05, **p < 0.01, ***p < 0.005.

The demonstration of the association between these SNPs and schizophrenia offers a large number of applications in the diagnostic and therapeutic fields. Thus, embodiments of the present invention offer the possibility of diagnosing schizophrenia by means of a biological test and no longer exclusively by means of clinical evaluations. Embodiments of the present invention can also be applied to diagnosing pathologies of the schizophrenia spectrum, such as, in particular, schizotypy, schizoid individuals, etc. Embodiments of the present invention make it possible to refine the criteria for diagnosing these pathologies, which is currently entirely established clinically. Furthermore, embodiments of the invention also makes it possible to demonstrate susceptibility to schizophrenia by means of identifying a genetic vulnerability in the families of patients who posses the identified SNPs in the RGS4 coding region and flanking regions. Once individuals have been identified as being susceptible to schizophrenia, the utility of prophylactic treatment may be investigated.

The DNA sample to be tested can be obtained from cells that have been withdrawn from the patient. These cells are preferably blood cells (e.g. mononucleated cells), that are easily obtained by the simple withdrawal of blood from the patient. Other cell types, such as fibroblasts, epithelial cells, keratinocytes, etc., may also be employed. The DNA may then extracted from the cells and used to detect the presence of SNPs in the RGS4 coding region and flanking regions.

Most preferably, the DNA extract is initially subjected to one or more amplification reactions in order to obtain a substantial quantity of material corresponding to the region carrying the RGS4 coding region and flanking regions. The amplification can be achieved by any technique known to the skilled person, and in particular by means of the so-called PCR technique as described above. To this end, embodiments of the present invention also relate to specific primers which make it possible to amplify DNA fragments that are of small size and which carry the RGS4 gene, flanking regions thereof, or portions thereof generated from SEQ ID NOS. 3, 4, 5, 6, 7, or 8. Portion of a polynucleotide sequence is specifically intended to refer to any section of SEQ ID NOS. 3, 4, 5, 6, 7, or 8 that can be used in the practice of this invention, such as use as a primer to identify the presence of SEQ ID NOS. 3, 4, 5, 6, 7, or 8 or variations thereof in a patient or a section of SEQ ID NOS. 3, 4, 5, 6, 7, or 8 that can be used to amplify the entire sequence. The phrase contiguous portion is meant to refer to a series of bases that are adjacent to one another within a polynucleotide sequence. In the context of the present invention, the word gene is intended to mean the protein coding region, the proximal 5′ and 3′ untranslated regions, as well as any distal and proximal regulatory domains. The phrase gene-coding region is meant to refer to the stretch of DNA that begins at the transcription initiation site and includes all exionic and intrionic sequences that encode a protein.

Embodiments of the present invention may also involve isolating DNA sequences and ligating the isolated sequence into a replicative cloning vector which comprises the isolated DNA of the RGS4 gene, based upon or derived from the cDNA of SEQ ID NOS. 3, 4, 5, 6, 7, or 8 and a replicon operative in a host cell. Additional embodiments include an expression system which comprises isolating DNA of the RGS4 gene, based upon complimentarity to SEQ ID NOS. 3, 4, 5, 6, 7, or 8 and operably linking this DNA to suitable control sequences. Recombinant host cells can be transformed with any of these replicative cloning vectors and may be used to overproduce the RGS4 protein.

Embodiments of the present invention also include kits that will facilitate the diagnosis of schizophrenia through the amplification of segments of the 1q21-22 locus. Several methods providing for this amplification are described including: at least a pair of single-stranded DNA primers wherein use of said primers in a polymerase chain reaction results in amplification of a portion of the RGS4 gene fragment, wherein the sequence of said primers is derived from the regions of the cDNA defined by or complementary to SEQ ID NOS: 1, 3, 4, 5, 6, 7, or 8. Similarly, embodiments of the invention also provide for a pair of single-stranded DNA primers wherein use of said primers in a polymerase chain reaction results in amplification of an RGS4 gene fragment, wherein the sequence of said primers is based on the exon regions of chromosomal DNA derived from SEQ ID NOS:1 or 3.

Various nucleic acid probes and primers specific for RGS4 (derived from or complementary to SEQ ID NOS. 3, 4, 5, 6, 7, or 8) may also be useful in diagnostic and therapeutic techniques and are included within the present invention. Among these are a nucleic acid probe complementary to portions or the entirety of human RGS4 gene as well as a nucleic acid probe complementary to human altered RGS4 gene sequences wherein said nucleic acid probe hybridizes to a variant of the RGS4 gene under hybridization conditions which prevent hybridizing of said nucleic acid probe to a wild-type RGS4 gene. Probes that are complementary to portions or the entirety of the RGS4 coding region and flanking regions that contain SNPs may also be used in these diagnostic tests. Any primer which makes it possible to amplify a fragment of the RGS4 coding region or flanking regions also forms part of the present invention. The primers that are used within the context of the invention can be synthesized by any technique known to the skilled person. The primers can also be labeled by any technique known to the skilled person.

The invention may also be practiced through detection of SNPs in the RGS4 coding region or flanking regions by a variety of techniques. The techniques which may preferably be employed are DNA sequencing and gel separation.

Any sequencing method known to the skilled person may be employed. In particular, it is advantageous to use an automated DNA sequencer. The sequencing is preferably carried out on double-stranded templates by means of the chain-termination method using fluorescent primers. An appropriate kit for this purpose is the Taq Dye Primer sequencing kit from Applied Biosystem (Applied Biosystem, Foster City, Calif.). Sequencing the SNPs in the RGS4 coding region and the flanking regions makes it possible to identify directly the SNPs that are present in the patient.

An additional preferred technique for demonstrating the SNPs in the RGS4 coding region and flanking regions is that of separation on a gel. This technique is based on the migration, under denaturing conditions, of the denatured DNA fragments in a polyacrylamide gel. The bands of DNA can be visualized by any technique known to the skilled person, with the technique being based, such as by using labeled probes that are complementary to the entirety or portions of the RGS4 coding region and flanking regions. Alternatively, the bands may be visualized by using ethidium bromide or else by means of hybridization with a radiolabeled probe.

In addition, measuring the expression of RGS4 message in peripheral tissue allows the diagnosis and determination of the susceptibility to schizophrenia in humans. As a matter of convenience, the reagents employed in the present invention can be provided in a kit packaged in combination with predetermined amounts of reagents for use in determining and/or quantifying the level of RGS4 expression. For example, a kit can comprise in packaged combination with other reagents any or all of the following components: appropriate detectors, buffers, deoxynucleotide triphosphates, ions provided by MgCl₂ or MnCl₂, and polymerase(s). The diagnostic kits of the invention may further comprise a positive control and/or a negative control as well as instructions for quantitating RGS4 expression.

Additionally, an embodiment of the present invention relates to ascertaining levels of the RGS4 protein. The level of RGS4 protein can be detected by analyzing binding of a sample from a subject with an antibody capable of binding to RGS4. An embodiment of this detection method utilizes an immunoassay. The sample from a subject may preferably be a biopsy of skeletal muscle, though any tissue accessible to biopsy may be used.

In addition to providing generally useful diagnostic kits and methods, embodiments of the present invention may provide a method for augmenting traditional treatments by supplying the RGS4 protein to a subject and/or augmenting the subject's medication, such as antipsychotic drugs, and providing an improved therapeutic outcome.

Further embodiments of the present invention may relate to the construction of an animal model of schizophrenia. Transgenic mice technology involves the introduction of new or altered genetic material into the mouse germ line by microinjection, retroviral infection or embryonic stem cell transfer. This results in lineages that carry the new integrated genetic material. Insertional mutagenesis occurs when integration of the microinjected genetic material into the host genome alters an endogenous gene resulting in a mutation. Methods of transferring genes into the germline, the expression of natural and hybrid genes and phenotypic changes that have occurred in transgenic mice are described by Palmiter and Brinster in Ann. Rev. Genet. 20 (1986) 465-499. Methods of foreign gene insertion, applications to foreign gene expression, and the use of transgenic mice to study immunological processes, neoplastic disease and other proliferative disorders are described by Gordon in Intl. Rev. Cytol. 115, 1989, 171-299 both of which are hereby incorporated by reference. A further example of genetic ‘knock-in’ technology may be found in Nebert, et al., Ann. N.Y. Acad. Sci. 919, 2000, 148-170 which is hereby incorporated by reference. The insertion of SEQ ID NO:3 containing some or all of the described SNPs into a mouse germ line may be expected to result in adult mice that may be used as an experimental model of schizophrenia. The insertion of SEQ ID NO:3 containing one or more of the variations listed in Table 1 with standard on:off regulatory domains will allow for the creation of mice deficient in RGS4 expression at specified times, and may be used as an experimental model of schizophrenia.

Having now fully described embodiments of the present invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention.

What is claimed is:

1. A kit for diagnosing schizophrenia in a patient, said kit comprising:
antibodies to RGS4; and
a detector for ascertaining whether said antibodies bind to RGS4 in a sample.

2. The kit of claim 1, wherein the detector is an immunoassay.

3. A method for diagnosing schizophrenia in a human, said method comprising:
obtaining from a patient a DNA sample comprising a RGS4 gene; and
detecting a variation in the RGS4 gene indicating schizophrenia.

4. The method of claim 3, wherein the detection of the variation comprises:
determining the sequence of the RGS4 gene in said sample; and
comparing said sequence to SEQ ID NO: 3, SEQ ID NO: 4, SEQ ID NO: 5, SEQ ID NO: 6, SEQ ID NO: 7, and SEQ ID NO: 8.

5. The method of claim 3, wherein the detection of the variation comprises:
isolating said DNA sample;
hybridizing said DNA sample to a hybridization probe comprising SEQ ID NO:3, SEQ ID NO: 4, SEQ ID NO: 5, SEQ ID NO: 6, SEQ ID NO: 7, or SEQ ID NO: 8, contiguous portions thereof, and a detectable label; wherein
the hybridization probe contains variations selected from the group consisting of:
a T at nucleotide base number 4121 of SEQ ID NO:3;
an A at nucleotide base number 4123 of SEQ ID NO:3;
a C at nucleotide base number 4368 of SEQ ID NO:3;
a C at nucleotide base number 4621 of SEQ ID NO:3;
a T at nucleotide base number 4790 of SEQ ID NO:3;
a T at nucleotide base number 4816 of SEQ ID NO:3;
a T at nucleotide base number 4970 of SEQ ID NO:3;
a G at nucleotide base number 5055 of SEQ ID NO:3;
an A at nucleotide base number 5295 of SEQ ID NO:3;
an A at nucleotide base number 5695 of SEQ ID NO:3;
a T at nucleotide base number 7375 of SEQ ID NO:3;
an A at nucleotide base number 7759 of SEQ ID NO:3;
an A at nucleotide base number 8596 of SEQ ID NO:3;
base numbers 9603-9609 of SEQ ID NO:3 are absent;
an A at nucleotide base number 9892 of SEQ ID NO:3;
an A at nucleotide base number 9963 of SEQ ID NO:3;
an A at nucleotide base number 10132 of SEQ ID NO:3;
a C at nucleotide base number 11056 of SEQ ID NO:3;
a T at nucleotide base number 11091 of SEQ ID NO:3;
an A at nucleotide base number 11106 of SEQ ID NO:3;
a T at nucleotide base number 11774 of SEQ ID NO:3;
an A at nucleotide base number 12143 of SEQ ID NO:3;
a T at nucleotide base number 12145 of SEQ ID NO:3;
a G at nucleotide base number 14367 of SEQ ID NO:3;
base number 17028 of SEQ ID NO:3 iS absent;
a T at nucleotide base number 17630 of SEQ ID NO:3;
a C at nucleotide base number 199 of SEQ ID NO:4;
a T at nucleotide base number 153 of SEQ ID NO:5;
an A at nucleotide base number 87 of SEQ ID NO:6;
a G at nucleotide base number 120 of SEQ ID NO:7; and
a C at nucleotide base number 221 of SEQ ID NO:8.

6. A method of determining susceptibility to schizophrenia comprising:
obtaining from a patient a DNA sample comprising a RGS4 gene; and
detecting a variation in said RGS4 gene indicating susceptibility to schizophrenia.

7. The method of claim 6, wherein detecting the variation comprises:
determining the sequence of the RGS4 gene in said sample; and
comparing said sequence to SEQ ID NO: 3, SEQ ID NO: 4, SEQ ID NO: 5, SEQ ID NO: 6, SEQ ID NO: 7, and SEQ ID NO: 8.

8. The method of claim 6, wherein detecting the variation comprises:
isolating said DNA sample;
hybridizing said DNA sample to a hybridization probe comprising SEQ ID NO: 3, SEQ ID NO: 4, SEQ ID NO: 5, SEQ ID NO: 6, SEQ ID NO: 7, or SEQ ID NO: 8, contiguous portions thereof, and a detectable label; and
further wherein, the hybridization probe contains variations selected from the group consisting of:
a T at nucleotide base number 4121 of SEQ ID NO:3;
an A at nucleotide base number 4123 of SEQ ID NO:3;
a C at nucleotide base number 4368 of SEQ ID NO:3;
a C at nucleotide base number 4621 of SEQ ID NO:3;
a T at nucleotide base number 4790 of SEQ ID NO:3;
a T at nucleotide base number 4816 of SEQ ID NO:3;
a T at nucleotide base number 4970 of SEQ ID NO:3;
a G at nucleotide base number 5055 of SEQ ID NO:3;
an A at nucleotide base number 5295 of SEQ ID NO:3;
an A at nucleotide base number 5695 of SEQ ID NO:3;
a T at nucleotide base number 7375 of SEQ ID NO:3;
an A at nucleotide base number 7759 of SEQ ID NO:3;
an A at nucleotide base number 8596 of SEQ ID NO:3;
base numbers 9603-9609 of SEQ ID NO:3 are absent;
an A at nucleotide base number 9892 of SEQ ID NO:3;
an A at nucleotide base number 9963 of SEQ ID NO:3;
an A at nucleotide base number 10132 of SEQ ID NO:3;
a C at nucleotide base number 11056 of SEQ ID NO:3;
a T at nucleotide base number 11091 of SEQ ID NO:3;
an A at nucleotide base number 11106 of SEQ ID NO: 3;
a T at nucleotide base number 11774 of SEQ ID NO:3;
an A at nucleotide base number 12143 of SEQ ID NO: 3;
a T at nucleotide base number 12145 of SEQ ID NO:3;
a G at nucleotide base number 14367 of SEQ ID NO:3;
base number 17028 of SEQ ID NO:3 is absent;
a T at nucleotide base number 17630 of SEQ ID NO:3;
a C at nucleotide base number 199 of SEQ ID NO:4;
a T at nucleotide base number 153 of SEQ ID NO:5;
an A at nucleotide base number 87 of SEQ ID NO:6;
a G at nucleotide base number 120 of SEQ ID NO:7; and
a C at nucleotide base number 221 of SEQ ID NO:8.


SEQUENCE LISTING (8 sequences)

SEQ ID NO: 1 - DNA, 2934 bp, Homo sapiens
gtacgctcaaagccgaagccacagctcctcctgccgcatttctttcctgcttgcgaattccaagctgttaaataagatgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaac

SEQ ID NO: 2 - protein, 205 aa, Homo sapiens
MCKGLAGLPASCLRSAKDMKHRLGFLLQKSDSCEHNSSHNKKDKVVICQRVSQEEVKKWAESLENLISHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLVNPSSCGAEKQKGAKSSADCASLVPQCA

SEQ ID NO: 3 - DNA, 20300 bp, Artificial Sequence
  misc_feature: A genomic sequence containing RGS4 nucleic acid sequence and sequences upstream and downstream to the RGS4 nucleic acid sequence
agttcaagaccagcctgagcaacatggtgaaaccccatctctactaaaaatacaaaattagacaggcatggtgatacacgcctgtaatcccagctacttcggaggccgaggcaggagaatcacttgaacctgctgggggtggaggttgcggggagcaagatcatgccattgcactccagcccaggcaacaagagcgaaatgtcatctcagaaaaaaaaaaaggcattttatatatatatatatatatatatacacacacacacacatatatatatacacatatatatacacatatatacatatatacacatatatacacatatatatacacatacatatgtacacatatatatacacatatgtatacacatatatacacatatatacacacatatatacacatatatacacacatatatacacatatatacacatatatacacatatacacatatatacacatatatacatatatacacatatatataatatacacacatatatatacacatatatacacacatatatacacatatatacacatatatatacacatatatacacatatatacatatatacacatatatatacatatatacacatatatacatatatacacatatatacatatatacacacatatatacacatacatatacacacacatagatatacatatatatacacatatatatacgtatatatatgtatatatatatgctccagagttcataagaggtagcagttgattaccactggggatagaggaaaagagagtttgacagcagtgtattgtgagaaggacatttcaggttgatggcaaatagtaggggaaatacataaatgtgtaataaaacctatctgtaaggtagttaagaaggtaacactatatatatatatagtgaaagcagtgtaaacctaaaggatgggccaaggatttaaatgttatagaagaatggctaagatgccaaagctcagtgtatgtggcagaggcatggtgtagggtgtgtccaggttcatatattgcattaagtgtgagaacaccctggagtatgaaccaagaaaatgcaaaagccagaagtgatggaggaaatgagacacaataatgaagatattgagaggagggtgtgggcctagagtgaagcttttcgtgccagtacttcttttgaaggcccagttctcttctctctcgggggctccttcatctctcatagagtccacagcttttaagggccaacacttgaggtcagcctggctctctcatttgagctggatagaacattttagagcaccatctattcttcaagaggaagtttaaaaataaaagaaccttgaagaggaaaaaatgtagacattcaatctaaccttttcattttactagccaaagctaaatagaatgcaggttacctgtttttcagccaggcaccatcatttcctaattgttataaaatttattattattgttgttattattattatttgccataagaagtttcccatatccttttagtataacaaaaacacaattcacaagcattataaaacccatggtgtctaactattaaaaaaattaagtggaacacacttgtcccagctactggggaggctgaggagggaggatcacgtgatcccagggggtcaaggttatggagagctatgattgtgccactgcactccagcctgggtgacagggaaagaccctgtctctaaaattttttttaaaaaaactaaactggttttattacagagattctggagacagctacacataaaagggtggtatgcctcatattagctacccagggaggtggaatgccaacttaggtggtgtcaccactattaaaaatgccccaaagcaatcaaaactgagaacttcctgggagcttagcattgtgcaaaagcagcacaaaacacttaaacaattcacagttgtgttggaatgggaaggcctggaaatataaaccaaagagtatattgtctaaattgatagagattacaattgcctgaaagaaaaagttgacttttaactagaatgttcagagtaggtttacagaagaagctcttaaactgggctccagtggatttgtcaatgctttggaagctggtggggtgggagggttggagggggcataaaaagtcatgttggtatgctctgctcaagtctccattctgtttccttttcctcttttcaatgtcatgtcccattatttcattatgggcttccctttatccaggatcaatatgccacctcttggttgtcttttacctacttctccacctcactatggaatcgtccttgggtagctcctgtgcttgggaacctgcacgggcacttttctgatgtcttgattccagctttactcctaaaacttaaatgctgaggggccaacaccatggcagtggtagggatgggaatgggggtcttgtaacacactacataaactacacgaaataaactacatgaaactcaacatgtttgcaagactcagttcacatccatgaggagctcatgcttctccctcctgctcccctagcacacatgattatctctatttggaaatgtttggcatttttggtgaagtgaatggttcaataactttctccaccatcagaacaaaagctctttaaggttagggatgggatcatacacacttcccttgtccaagtccccatcaccccttatctagacaattgctacagtttcctacacactcttctaacctcttgcagtctattttcataaaacagctagagaactttgagatgtaagtcaaaaaatagaacatgtcgctctttcccattgtttttgaaataaagttcaacccccttaccagggtcaacaaggccctgcaatgatttggtcctgttaaaaattctttagccttaactcatgctgttcttccttacactcactgcattctagccattgaggtttctatgcatcaaactttttttggtcccagcactgtgcacatccttctgggtagaatgccccttgatttgtataattagcacctccttcatcatttaggtcttagtataactactaccttcttagagaagctctgcttcttcatcctataaaaaagtaaaattccttaccctgttattttttaagtcatccgtgtttcattctgttaaagttcttatcacaatttatcattattttatttacagtcatgtgccacataacaatgtttcagtcagggatagaacacaaatgtatctggccccataatattataagctgagaaatttctattaactagtgatatcgcagccatcataagtgtaatgcaggacattaccttttctatgtttagatatgttagatacacaaatatatttcattgtgttataatttcctacagtattcagtacagtaacatgctgtacaggtttgtaacctaggagtaataggctataccatacagcttaggtgtgtagtaggctataaccatctaggtttgtgtaagtacattctatgatattcccacaatgatgaaatcacctaactacacatttctcagaatgtttcactgttgtgaagtgacccatgactatattttcctatatacttgatatttttgtgcatctgcccatgagaatgtagtgtaagatcaaaggatgcaagaatgggttctatccagtatagtacccactacactggtggatgtcaatatgtatttgttagattaatatctcaagaatgagcacctttctcagacacataaaagatgctcaatataaaagtttgttgaactgaacgttattggcaaatgtaacatgatcggatttaaagaggagcgaaacagaggtctggctcaaacaccatacttctagagtgcataagaggtagcagttgattaccactggcgacaggagaaaaaagagcttgaccgcagggtactgtgaagacatttcaggttgatggcacagaacaggggaaatacataaatgtgtgggaatattcagtggtctgggatgactacatagtagaatataatgaagaaaagagtggaagggaaagatgaaaagttggaatggggatgaattatgaaagtaccagaatgttatgctaaggaatctagattttaaaatgtgagggcaaattgaagtcctgggcacgttacaaaactagaggtcataaagtttaccctaatttaccaagatttcctagaggatctataattggaatccagatctgcctctctgtaaagttcaagcactttccatgacaccatactgtttctttccacctgcacaatgcaaatgaactcttatgaaactgctgtttctatcctgggctaaatgttgcagaaaaaagatttaatctttgggataaggctattttgggttttctcctacttcttgggaaacaaggttttcttcccctggctaattaagtgtggtattgttcttccagggaaatcagtgatgcatcacctgctgctatcaaatgtcagggttggagttcctgatttattgcatgtgcccacaaagcttggtgcaaagaattggacacatttcccaaaagtaagacatactgggaagtccctgtttaccttcctggtatacagcatcctccagccccatatctttgctttttagtcctaaaaatcaataactgaactctcattgatgtctaggccattgtagtaaacaataaagaaggagggaggcttctgacaactgagaggaaattgtcatctgaagtggtgcaagcacagcctggggctgagccttggcctacatcctgcccaagtggaggatcagtgccccatttaacatctggtagaactaaagaacgcaacgcctgccacaatgacttatttccctgcatttgataccgtcaatccttgagaaatgttttcttttgttctccctgagcaaaggttggaaaaatttgaaatttacctagagaccacacatagttcacatcctgctgtgtggctgaatgtctgccccccagtaggaaacagttcttctaaagcctattgtcaacaataccttccagatgttagcattttacaatttaaggaacttaaaatagccttcaaactttttgccagtttctctgatatccaatctattcttttactctgcctcccaagctttctttctagaatgctaacctgatcggcttaagtacttgaactacctcttctcctccattaactacagagtaaattctggtcttcagagtaacaagaaacaccctttagttctcagcatattcgtgcaccttcatttatctctccttctctctcaaagctgcagtaggggtgaaaacgtgtgatacattttctcttccatcataagggtcgcaaccaaaactcctatagtaaaagacaggttaataagagcaaaacctaacaaatttatttaatcaaagttttacatgacatgggagtcttcagaaatgaagacccaaagacccaggggaaactgtctgttttttttgctgaggttcgatgaagaatggatagcatgtagccatgtagattagacaaaaggatatgatctagtggtaaaggactcagggggaaacacagcaaggcctgtctattcagattcttcttgatctctctctctctatgtatagcattctttcctcctgagtatggggcaggactcttcttcaatgagggtcttcaagggagaagggagaaagtggcctttttagattttatggcttgcttcggggaagaggagttctagtttctatgacccatcttggggaagaggaattctggtttctgtgacttgctttcatgaagaaagaggagtaagaggcaggagggcaggagatggtcagaaagagacttggctgcttctgagggcttccgctctcctttagttccaagtacttcttagcataccaaagcactatactttggcatatggttttctgagctctaacactgcaatcatgctaaactcctctatgaccttcaaacattccacttgcttttattctttatggttgtgatggcatagaggtcaatagcaaagaccctggagtcccactgtctgagctggcataacattactaccacttaatcaatgtgtaagctcaggtaagtacttaagtcctctatgcttcatctgtaaaatgagaatcattgaagaacattctctcaggatggatcatgaggaataagtgaattaactggcatatagtgcttaaaccagtgccttgctcagttagtgacagataaaatcatctgttattactgtgcccactattgtgatgctcttctcttctttgtacaacgactacatctctatttatcattttagggtctccttgtgaaaaaccactccagattcaaaagattgagtttaatctctatcctctgtgctttcctggagttttgtaaagtaaatcttcacttgacatcatggataggttcttggaaactacaacttcaagtgaaaggacataactaaaccaatttttttctcatcaacgttataatgaaatggcattgatgaaatgatggcattcaaggacctgctgtaccttgtttcacttaaagtcactgtttccaataatctattgatgacattgaggacttactatataataataaatatatatataatcgacgaaacaggaatcaaactgctaactctgctaactggtctccctgcttccacactctgcccactcatctcagtctttctttcacaagagtcagaatgatcagatgagacccctcctctgcttctgtttcttccatggatttccactgcactctgataaagtccagcctcttgaccacagcctacaaatccttgcacgatctatcgtttacttttccatctccttttatgctactttcatcttgttctcaattctctagctatgctggccccttcttgttctttcccatttttttttaatttttaaaatttgtatatatttatgggttataagtgaaatctttttagatgcataggttgtatagtgataaaatcagggcttttagggtattcatcacctgaatgatgtacattgtaccccttaagtaatttctcaccatccgctgacttcttgccccctgggtattcatcacctgaatgatgtgcattgtaccccttaagtaatttctcaccatccgctgacttcttgccccctgggtattcatcacctgaatgatgtgcattgtaccccttaagtaatttctcaccatccgctgacttcttgccccctcatccttctgaggctccattgtccatcattccacactctacatctatgtgtacacattatttagctcctacttataagtgataacatgcaatatttgtctttctgtgtctgtcttgttttacttatgataatggcccccagttctatctaggctgctgcaaaaggcatgatttcattcttttttatggctatgttctttcccaatttagataaagaacactcgcacttgctcttacttctatttggaatactaattcctaggcttcttgcattgctttctccttctcacccatcaaatctcattttagataccacctcttcaaagagggctttcctgaccaccttggctgaattagcccttcaccatctgattactctctagcacatcacctgcccattttattcatggtacaggtcaaaatctggaatcacctgatttgtttattttctgactccttctactgagatgaaaactctactagagcggagattttatctgcttgtatcaggtactgcttcaaacagcacctgatacagagtaggtggtcaaaagatatttcttaaacaaatgaacaaataaaaagtagatcttttgagagtaaagctcttccacactaccagagtcattcaggaatgacaaatcatagaataacagaatttgatgctttgtgcatatcagagaaagaaggtggaaggttgtcaaggtatcatgatgtaccagtcctcgcctcctcaaacacaatctgcaagtcccacagtgaaaaagtaagttaactcatgtgaagcgttttacaaacacttttttaaaagtcttaaaactcctaagaaagcaagatttaatagtcaaagaagtgagtaaacatgaaatgcctgaacagagtaatgagctaagcacaaagttagagacatgttagttaatatgtcttgaaagcagcagctcctgctttcaaggagcaagaacaaattgggcaagtgaacactccttgaataaaatgtgtaaaattaattttgggttatgttctatactgtgtataatagaatgataaaaattatttgactagcactttgtagtttagaaatatctctatttacacagtttaccttatttgataagactgttgagtgatgggatagcatggtggacaatccacataactgagtatcgagacacctgtatctggacccagctctgttagtaagaagctgtaacctcagcaagtcactttctctttctgggtctctatttcctttttggtgaaatgagagtgttaggctagattgcctttgaagtcccattttgtctttaaagtcccatctattgcagtgatttatatttaactcatgacaaatcaggcttctcttattctaagtgcaagacataaaacttttattgtggaatttcaggcatcagtaaatctttttgggtactcacttatgttcctgaaatcaatctatttgagtgatcactcttttaggtgcccaggtaaacaaagaaggccatggtctttctttgagtgaccttctttcccttttaattagtctgacctctttaatgtcagttctgactgattcatttccctggtccatcttccttggtctgagggccttcctagtttcatattgcacttcagttccttccacaccaccatcaaggatggctgtcaacattcatttgttctatgttataattcaaggaaaagttgcccagtagctaatccaataaatgccctcttatgggcggctagagactttttcctataatttaaatgcatcttctgtagattatggtccctccaccactttacatttgtctgctgtctccttgctctgctagtcatggaacgtgttggtagtgggggcagtgtgggatgttcaagggcacgtattgggtagggccacatatgggcattgctttgtgccattctttctatatttttggtattttgcatctcactggaacccaactatttttcatctcttccacctaaactatttgatgcctctgtttcttatatataaagtatagctcactgtagcctatgatcaggaacctatctgctttctaaatgaaagctgttttggtcagatctagcaattaattcccttcttccacttatagctttcctctgtaactctggtgtaggtatttggtttatggctataagatgtgaaacacctgaatgattctgtccatgcaggcatttcagttcatgatattgtatgtaaaagatactgattgtctaggtgttcagaaacacctatagggcttaatattcttacaatcagtttgaaggctggtgatacgcaaagcaaactacatatttttctgcctgctctctctctttctctctacatctctctttctttatcttttgaaatatcagtttggagacttagaattacataagacataaacccatttgatataagaattgctgtgtatatttgctcatctactccctcctttggtcctcgagctgccggtttagactttttacaggacgcaggcatgtgaaggagaaactgtcagtgctaggctgaattctgttgttaccaagatttctagaaaagtattcctcagtcaggttgattacagatatagcaaatctatttttcctagggtagtttctgtatgctgccgggcttataactgtctgtcatccagctatttctctccaccttcttgtttgcataacaaccaaggcaacttccgcaaatcactgcgtggagacgatgatcctgccagctcccttttggaaatcgtgaggatcagatcttggaccatgtataatatgatgcttctaatccaaaagaggaaaggcattgggagtcagctcctaagtaagctccagaattcctgctggtacttttccttccaggaagcaacttccttgatattttttttttacaggcatatgaataaaaactatattttgcagcattgtacactttttttccttttctagaaattctaaacctctgacattggtggagacattgagtacattttttcccatatccctacttttcagaaggattttctctgctcgttcacttaacattgctgatgcgtcagtcttttcttcctcatctctttcaggggctggagaggcagagggagacagaggagctggtactgcagagcggtcgtctgattggctggacggtcgtagctgggctataaaagagacccctacaggcttagcaggaagacgctcagaggattctgacaatatctttaccggagaagaggcaaagtacgctcaaagccgaagccacagctcctcctgccgcatttctttcctgcttgcgaattccaagctgttaaataagatgtgcaaagggcttgcaggtctgccggcttcttgcttgaggaggtaagattgctttcagccattaaccatattaaacttttggctagactttctcagttatttacatgttgtacttactaacctagttctgtgcaattagaaacagtgtggtcaggagagcacgactttctaactttcctccaagactagctagatattgtgacttaagacatgtgctccccaaatttcagcccttatgtgttgttttgtgtgacctcagttttgagaactgttctattctttaagccaggtctaagaaagctagttttaattaagaagcgagatgaggtttgaggctatgtacagtgatctgtaatatctccatctgtgattactactgctatttgagcatccctggagtacatagaagcctggctctgggctttctgattgtatgctacaacttgtttcaggaaaggtaccccagaatgaggtttggctccatcatcagaaaggcactatgctttccgtgtggtggtgcagtaactttcactctctatgttcttataagcaaatgttacaatgagatatgagttttaaagccagatcttccttatctctctgccccatctctagttcttgaagtgtctcatatgagtttggttgagaaatattgatcattacaaatcagttaatagttttgtagaagatctcatcttaaagacattgttttgttaatatactcccttgatttttttaaaagaccttacagacatacagctattcatttgtttttggtttgttcaaaaaaggtataaagaaatgcattcagagaaagatcatatattagccagttgaaaattaaacacaaaatgagtgcatattacattacttaatcttgcagtcaaaggtaaaaagtcaacctaaaggtatactacctgctttcttatcgcactgcaaatagaaattaccacaaattttattttggaaataatctcagaaaacataattttttatgtactattaaaacatttactttccaaatattctgtcattcaggagtatggaagtatcgatggcttctttaaaatgaagcaggagggtctggcagagagtatctatgaaataagttcctctgaccttcacgcttaattttctgaatggagtggagcaaattacttcaagcttcacttaacttgcatatgaaatgaaccgtacaaaaatacaagagtgtcaggagaaagttatgctctggtaaatattttgcaaaacagataaaagataatactagagctctgtcctcaaagagttaagcagctaatctaaggaggtaaactctatgtcagcaggatgaactgctcttccctttcctcctcaataaattgcaaatcatctagtccaacatctttaccaccagtgcctgaggctccagaggagccattgccttctcaaggtcacataggtggtgggtgagttaggaccaaatctagaattcctgactccagtaacttctgaagtcattttgttttttatttttatggttttattataagaatacttgctaagcacacttaccccctgcattgattaataactctaggatctcaggtggatccagcacatagaaatatgaattcgtttctatttggacttcatgatatatttacattatcaccttggaatcaccctaacattcaggattgtatcttgttataatcaaaaaggatgttgcatcccctgaacagtcatcagtcagggaagcagaggagggaaagtaatcttgcgaggaagagaaaatactatttaagggacagtcagagaacataatggaattcaaactttctgggaaaacctacatacataaatgtattagtggccatcctaaatgtctttatatctttgaggctttattttccctactccaaatagacacatttagttattcatttcttttaaaatggtatttctctttttaaactatttcttgacttttttaataaaaagagatgcaagcaagaggatatttaataaaaagtaagagagttgagcttaaggcttattaaaagaccccctttttctagttagtcaggagctctaatgtgccctggctacctattaaatggtggcaataaactggaagctcagtgatgactctagcctgcttctcctaatagctgttaagcctcaaatgccctttagagtgtgtatgtcctttaaagtagctattaagaaggaaagcagcagcagcagatattgtctagaaagaagccccaagaagctgaggtttcagcttgggcatttgttttcgccatcccatgctccatttccctctgctggaactgtgcacctcagtgtattctccctctatacctcacagcaggaactgcttgccccccccccccccccccaacatacatggctggaactgaatagacttttactttcccgaggtgcttctacagttccctctgccagcaggggaacagatggaaatagcaatcacctgccagaaggtggcgtgcagcaaggatgtgcatcttttgccgctactgctttctgattcctaaaaattactcagagatcactcatgtgttcagtgattcaggttctgttgaagataccaaagatattcggttggtcaaaatgacgggcatataaaggcttctcaggtttctgaggtaaactgaagggtcagaattccagttgtggatgaaggaaatggtgttatgactgcctcaaggttttgtagcaagtcatagggaaccaagaggaatcttgttttcctcagaggtcatgccaactccaactcccgttccctaaactgtctctgagccatagactagtaatggactcttcaagctctaccattaggtatcttttaaagaaagctggttattactatttattcatttttttctcttctgtgcagtgcaaaagatatgaaacatcggctaggtttcctgctgcaaaaatctgattcctgtgaacacaattcttcccacaacaagaaggacaaagtggttatttgccagaggtaagagaaaaggccttggtgaagatgtacttagtattaactatctgatgatggggatgttctgtgagaaggaacttgtgctcctagttaagccagatttggatcaagatagcctccattttcatggagatcataactacatttgaaatttctatacatttagtgaaaaactgccctcatcaataacatattttgtcataacgatggaaaataaaatctttgccttcattcaggatcttagatttcttgccccaatttttttaccatggcattccaattattctgtttctctctattttttctagagtgagccaagaggaagtcaagaaatgggctgaatcactggaaaacctgattagtcatgaatgtaagtctgacagcaacctgggatgaggtactctggataagacaagttatattatgctggtctaatagaaactgcagcaaggcctggcttctttctgatgttcagactcaggagactctttaggtcttaaattcagtctgtttaaaattttaatatgccctagagctttgtgatatacaatgaaaagtttatgcaggaaccatgtggaaaaccatctctctcatcacaaggaaaaacggaagagagaaaaaaaatgataaatatcaataccttcttgcaaaatcaatctcagtttctctttcccaaattgaccttggtaattgatagctgcataggcatttcagaagcaaaatacttccttgaaagaggcttccaacttgagtaagaatcattaggtagaactgggaaccactggatatcaaacacagattagggttacctgactccaggtgacttgaaaaaagcaggggaaaaagggattgcttgaatccatgctttatcccccaagtacctcagctttatgtgaaatagcatatccaagaggccaaccagtgtgatgacaactgtggtcctttctcctgtatcataggtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtaggttttttatggatacataaaaattgtacgtatttatggagtatgtgtgatattttgatacatgcatacaatgtgataacaatcaaatcagggcaattgctatatacatatctcaaacatttattatttctacgtgttgagaacattccaaatctcctcttctagctatcttaaaatatacaataaactattgataactatatcaccctaatgtgctatcaaacactagaacctattccctctacccaactttctatctattccttctacccattagccaacctgaccaaaaaggtaagcttttatggcagagaactctctggatcttagtgaaggttcctagaatagtggagctgactatcataatcttgacaaccccaaataaatcagttttttaaaaaatctcttttatccatgtggcttaccataacctccctgcatgaatttttctgatgaatctccccaatttgttagacagaacagaagatcttgccctgctctctctaaagcagaaaggttcattctgaacctttcatactctctcacatgtgccaaggaggaccccaatgtcacttttgttttttgcttctgaaatacagagggtgcactgccacttacaagtcactacaaagcatacaggcttgcatcctcaacagggatataggtctaatgaagccttggcctttgcccctcaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaactatttgcttggaaacacattaatgatccagagacatgctatgagaaacatcagggtgtagggtgactttagaaaaatactcatactgagtctttaatccctcctgtgccagtgaactctgggaaagaaagtacaaactgaatattgtttattctttagttcatgccactgctctgcttggctctactcatagaaccaaggcaatcttagcttcagagactgcaaaacagattaagtgatttgcttgcagattctcaatcaattttcaagggatagagttcaccttccagagccattcttttatttccagttacccgcctgtttgagagatgatagagcagtgggaaattgagagagttgaaaggagctatagattcttacccaaacttcaaaaatccttccctcccttttgttaattctctttcctggaaaagaggtcataaaatgttcacatcctcagtaataggccctgtgctgtgtctattatgtcatgagactcccatttcctgacccttctttcccattgtaagagtagtagttacaaggtgttaaggatagatgatcttcaacacttttgagaaatagatccatttacggatctggtaaaaactatggaccgaaccatcttttaagaaaaaaattcagagaggaatctaaattttgtgtgctttgaggggaaactctcagaatctcccctcaaaactatcattcttctcttatactatagatgtgtcagactctcactgggactgtatagttgctgctccctgtatttgataatatctatcaagaactgcagggtaattcaaagtcacgctattagcagcaagtgtgagcagtgttggtttccccagtctctacatccctcatcctttctttcttctttatggttgtctattaaagaaataaaaaaaaatattggctgaccgtttttctgaagataatgtatatcaaggaccaccttttgaaaaacactcattattcgagaacaaagacacaacatacgagaatctctgggatacattcaaagcagtgtgtagagggaaatttatagcactaaatgcccacaagagaaagcaggaaagatctaaaattgataccctaacatcacaattaaaagaactagaaaagcaagagcaaacacattcaaaagctagcagaagacaagaaataactaagatcagagcagaactgaaggaaatagagacacaaaaaacccttcaaaaaattaatgaatccaggagctggttttttgaaaagattaacaaaattgatagactgctagcaagactaataaagaagaaaagagagaagaatcaaatagacacaataaaaaatgataaaggggatatcaccaccgatcccacagaaatacaaactaccatcagagaatactataaacacctctacgcaaataaactagaaaatctagaagaaatggataaattcctcgatacatacaccctcccaagaccaaaccaggaagaagttgaatctctgaatagaccaataacaggctctgaaattgaggcaataatcaatagcttaccaaccaaaaaaagtccaggaccagatggattcacagctgaattctaccagacgtacaaagaggagctggtaccattccttctgaaactattccaatcaatagaaaaagagggaatcctccctaactcattttatgaggccagcatcatcctgataccaaagcctggcagagacacaaccaaaaaagagaattttagaccaatatccttgatgaacattgatgcaaaaatcctcaataaaatactggcaaaccgaatccagcagcacatcaaaaagcttatccaccatgatcaagtgggtttcatccctgggatgcaaggctggttcaacatacgcaaatcaataaatgtaatccagcatataaacagaaacaaagacaaaaaccacatgattatctcaatagatgcagaaaaggcatttgacaaaatttaacaactcttcatgctaaaaactctcaatcaattaggtattgatgggacgtatctcaaaataataagcactatctatgacaaactcacagccaatatcatactgaatgggcaaaaactggaagcattccctttgaaaacgggcacaagacagggatgccctctctcaccactcctattcaacatagtgttggaagctctggccagggcaattaggcaggagaaggaaataaagggtattcaattaggagaagaggaagtcaaattgtccctgtttgcagatgacatgattgtatatctagaaaaccccatcgtctcagcccaaaatctccttaagctgataagcaacttcagcaaagtctcaggatacaaaatcaatgtacaaaaatcacaagcactcttatacatcaataacagacaaacagagagccaaatcatgagtgaactcccattcac

SEQ ID NO: 4 - DNA, 480 bp, Artificial Sequence
  misc_feature: A genomic sequence occurring upstream of RGS4
ggattaatcatgacaaaagtaatctaaatctcgttaagactacttaatgatcaatctttccctctgttttccctgactatagggaagtgaattgccccaatccttctctatcacccccctgcagccatgccaatgccttacctctgttatattcagccataggggaagcttattctcatagaatcaggggttggcatgtagtcactagctattcttggtgagactagtgaagatgagtgaaggaaaatattgcataggtgaaatctcataggcacaaataggtgtttgtgagagtaacaataaaagaaagtcattcccatactctagtagatgactcattttctcctcattttttttttttcaaggcgttctctacaacggttaacctagtaccaaaaatccttctcttttttcttggacaaatcctgttcaagttagcatggcatttactacgtccaagacattgtccagatgctgtgg

SEQ ID NO: 5 - DNA, 420 bp, Artificial Sequence
  misc_feature: A genomic sequence occurring upstream of RGS4
agagaaagaaaggcaggcagcaaggagaaaaaacattttttaaaaaaagaaaattaaaatccatgtaatgtctgatatctgttctgctgtatgtgtagatctttccatataccaactcattagccttattttacaggtgaggaaaatgagaccgagagtccttcttacttgaccaagttcacacagcaagatcacacatggtagaaccaatgttagaacctaggtgtatacttgctcattcaatatgtacaataattgcaaaagtttccataggtcttattatatatcaggcactataaatgctatgcatgtgtcaactaatttaaacctaagcaatattataaggaaggtactattatagaaatctcagccttacaggtaagggaacaggaataaagagatgtgaggtaatggcccaag

SEQ ID NO: 6 - DNA, 360 bp, Artificial Sequence
  misc_feature: A genomic sequence occurring upstream of RGS4
ataatctcctttcaagtttttatcctgtcacttgctagttgtgtgatttgggacaaatcatttaactccttgtaaagggagagaaggaaggctgtaaaaaaattaagtaataaaaagataaactccttgtggtatattttgttattgttcaaaaatatttattgcccctcttaggatgtcttaggtcattcttgcattgctataaagaaatacccaagtctgggtaatttataaagaatagaggttaaattggctcacagttctgcaggctgcacaggaagcatcccactggcgtctactcacttctggtgaggactcagaaagcttttgcttatgacagcaggctaagtgagagcaggt

SEQ ID NO: 7 - DNA, 420 bp, Artificial Sequence
  misc_feature: A genomic sequence occurring downstream of RGS4
catggtatttttactacccattgccttctaggaaagggtataacaaataggaaatattaatatttttaatgcctttgagggtgttaaaaagcacaactctaaggactgtttgtaaattccaggtcaaatgttgtttctccttctctatttcctaccttggtgatggcctgatcttatatggagtcactccaactagaaaccacagaatcatccctagttcctacttctgactcactccatacactcaaaagtcacctgactctgcagaatttctctagaaaaactctatgaaaacctattcctgcctctccacctgcatagatgtagcttcatccaggctcttatggtgcatggcctcggttactgccttatcctttctactggcctctcaatctcccatctgatacccattaatgtact

SEQ ID NO: 8 - DNA, 360 bp, Artificial Sequence
  misc_feature: A genomic sequence occurring downstream of RGS4
ccaaatactttttaggcacactgggaagttacattgtttcttgcaagtgacaggttgtcctttaattagttctttctctcaaaaagagactgctgactccaaactgggaagaaacccactcaccagcaaaatgctgctgaattcactctgatagttttctaatctctcatcagtagatgacaataatgaagccagtattgttaccacaagactcagatatgtctatcacccaagatgatttctctttaagacgcaataaaagggaacttttctccccatttattagcaactaagatgaaatgagagccagagaaataaagtgaggaaggaaagagaatttactacctttacaagctgaaa